(19) United States
(12) Patent Application Publication
(10) Pub. No.: US 2010/0256098 A1
(43) Pub. Date: Oct. 7, 2010

(54) COMPOUNDS FOR INHIBITING WIP1, PRODRUGS AND COMPOSITIONS THEREOF, AND RELATED METHODS

(75) Inventors: Ettore Appella, Chevy Chase, MD (US); Daniel Appella, Rockville, MD (US); Stewart R. Durell, Bethesda, MD (US); Jeong Bang, Chungbuk (KR); Hiroshi Yamaguchi, Nagasaki (JP); Qun Xu, Rockville, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: The United States of America, as represented by the Secretary, Dept. of Health and Human Services, Bethesda, MD (US)

(21) Appl. No.: 12/675,167

(22) PCT Filed: Aug. 29, 2008
(86) PCT No.: PCT/US08/74864

§ 371 Date: Mar. 25, 2010

Related U.S. Application Data
(60) Provisional application No. 60/969,258, filed on Aug. 31, 2007.

(51) Int. Cl. A61K 31/675; C07F 9/06; A61P 35/00; C12N 5/071; C12N 5/09

(52) U.S. Cl. 514/91; 548/413; 435/375; 435/366

(57) ABSTRACT

The invention provides compounds useful in inhibiting the activity of a Wip1 protein in a cell as well as prodrugs thereof, related methods of use and compositions which include the aforesaid compounds and prodrugs thereof. The compounds comprise a ring structure having at least five functional groups bonded thereto, wherein each functional group is bonded to a different ring atom, and wherein the at least five functional groups comprise: (a) first (R₁) and second (R₃) moieties each comprising a phosphate group wherein these first and second moieties are separated by at least one ring atom; (b) first (R₂) and second (R₄) hydrophobic groups, wherein the first and second hydrophobic groups are separated by at least one ring atom, and wherein the first hydrophobic group is bonded to a ring atom located between the ring atoms to which the first (R₁) and second (R₂) moieties are bonded; and an amide or carboxylic acid (R₅).

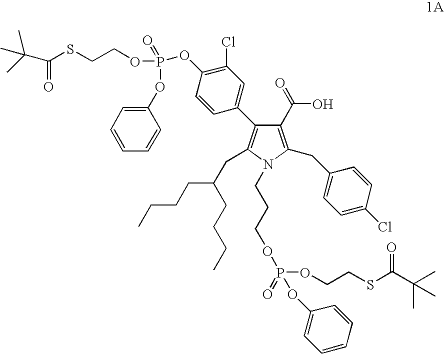

### RELATED APPLICATIONS

The application claims priority to and the benefit of U.S. provisional patent application No. 60/969,258, filed Aug. 31, 2007, the content of which is incorporated by reference.

### BACKGROUND OF THE INVENTION

The wild-type p53-induced phosphatase 1 (Wip1), also known as PP2Cδ or PPM1D, is a member of the protein phosphatase 2C(PP2C) family and is expressed in response to ionizing or ultra-violet (UV) radiation in a manner that is dependent on the tumor suppressor gene product p53. Wip1 inactivates the p38 mitogen-activated protein (MAP) kinase through dephosphorylation of phosphothreonine in the sequence of its regulatory site (Takekawa et al., EMBO Journal, 19(23): 6517-6526 (2000)). Phosphorylated p38 MAP kinase phosphorylates and activates p53, thereby causing cell cycle arrest or apoptosis in response to DNA damage (Sanchez-Prieto et al., Cancer Res., 60: 2464-2472 (2000), Bulavin et al., EMBO J., 18: 6845-6854 (1999), Kishi et al., J. Biol. Chem., 276: 39115-39122 (2001)). Thus, Wip1 controls a feedback loop in the p38 MAP kinase-p53 signaling pathway (Takekawa et al., supra). Wip1 also interacts with a nuclear isoform of uracil DNA glycosylase (UNG2) and suppresses base excision repair through phosphothreonine dephosphorylation of UNG2 (Lu et al., Mol. Cell, 15: 621-634 (2004)). It also has been reported that Wip1 dephosphorylates specific phosphoserine residues of the p53 and Chk1 proteins (Lu et al., Genes Dev., 19: 1162-1174 (2005)) and specific phosphothreonine residues of the Chk2 protein (Fujimoto et al., Cell Death Differ., 13: 1170-1180 (2006)), suggesting that Wip1 may play a role in controlling cell cycle checkpoints in response to DNA damage.

The foregoing studies suggest that the Wip1 protein is a promising target for treating various types of cancer. Recent studies have identified inhibitors of Wip1 (Belova et al., Cancer Biol. & Ther., 4: 1154-1158 (2005), U.S. Patent Application Publication Nos. 2004/0167189 and 2005/0037360, and International Patent Application Publication No. WO 05/089737) or the related phosphatase PP2Cα (Rogers et al., J. Med. Chem., 49: 1658-1667 (2006)) by screening libraries of small chemical compounds or by computational analysis; however, the mechanism of these inhibitors has not been elucidated. In addition, it has not been demonstrated that these inhibitors exhibit specificity for Wip1 and not other PP2C enzymes.

Thus, there remains a need for compounds and compositions capable of inhibiting the activity of the Wip1 protein for treating certain types of cancer, and methods relating thereto.

### BRIEF SUMMARY OF THE INVENTION

In one aspect the invention provides compounds comprising a ring structure and at least five functional groups bonded thereto, wherein each functional group is bonded to a different ring atom, and wherein the at least five functional groups comprise: (a) first (R₁) and second (R₃) moieties each comprising a phosphate group wherein these first and second moieties are separated by at least one ring atom; (b) first (R₂) and second (R₄) hydrophobic groups, wherein the first and second hydrophobic groups are separated by at least one ring atom, and wherein the first hydrophobic group is bonded to a ring atom located between the ring atoms to which the first (R₁) and second (R₂) moieties are bonded; and an amide or carboxylic acid (R₅).

A related aspect of the invention provides prodrugs of the foregoing compounds.

Another aspect of the invention provides methods for preparing the aforementioned compounds and prodrugs thereof.

Also provided as a further aspect of the invention is a method of inhibiting the activity of a Wip1 protein in a cell. This method comprises providing a cell comprising a Wip1 protein, and contacting the cell with at least one of the inventive compounds and/or prodrugs thereof, wherein the activity of the Wip1 protein in the cell is inhibited.

Formulations comprising at least one of the inventive compounds and/or prodrugs thereof in a suitable carrier, which formulation may be administered to a mammal for the treatment of disease or condition, also are contemplated and provided by the present invention.

### BRIEF DESCRIPTION OF THE SEVERAL VIEWS OF THE DRAWINGS

FIG. 1 is a graph of the relative Wip1 inhibiting activity of compounds 7, 8, 16, and 24 of Table 1 at a concentration of 0.1, 1.0, 10, and 100 μM.

FIG. 2 provides a Western blot analysis concerning the Wip1 inhibiting activity of certain compounds.

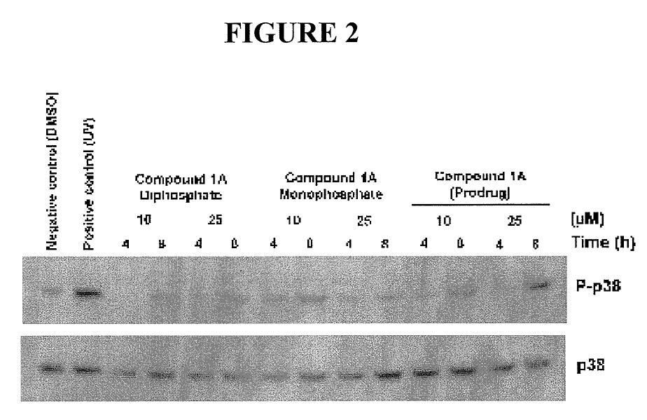

### DETAILED DESCRIPTION OF THE INVENTION

In one aspect, the present invention provides compounds which are capable of inhibiting the enzymatic activity of Wip1. Generally, the inventive compounds comprise at least one ring structure, which ring is desirably aromatic or heterocyclic and more desirably both, wherein the ring comprises at least five functional groups each bonded to a different ring atom. These five functional groups comprise: (a) first (R₁) and second (R₃) moieties each comprising a phosphate group wherein these first and second moieties are separated by at least one ring atom; (b) first (R₂) and second (R₄) hydrophobic groups, wherein the first and second hydrophobic groups are separated by at least one ring atom, and wherein the first hydrophobic group is bonded to a ring atom located between the ring atoms to which the first (R₁) and second (R₂) moieties are bonded; and an amide or carboxylic acid (R₅).

It should be understood that the invention contemplates that the groups described herein, e.g., R₁, R₂, R₃, R₄ and R₅, may be substituted or unsubstituted, despite this not being explicitly recited in the description or claims.

Prodrugs of these compounds, also contemplated as an aspect of the present invention, will be discussed in more detail below. References herein to the inventive compounds, including but not limited to uses and formulations thereof, should be understood as including these prodrugs unless excluded either expressly or by context.

The ring structure contemplated by the present invention may be any one which comprises at least 5 ring atoms that are capable of being substituted with the groups described herein, e.g., R₁, R₂, R₃, R₄ and R₅. Suitable structures include cyclic, bicyclic and tricyclic ring structures, such structures exemplified by benzene, naphthalene, anthracene, and the like, as well as heterocyclic ring structures such as pyrrole, quinoline, isoquinoline, indole, and the like.

In a desired aspect wherein the ring is heterocyclic, the hetero atom therein may preferably comprise nitrogen or sulfur. Even more desirably, one of R₁, R₂, R₃ or R₄ is bonded to the heteroatom, with the latter most desirably comprising nitrogen. Preferably, the ring is 5- or 6-membered and heterocyclic, more preferably comprising, in the case of a 5-membered heterocyclic ring, R₃ bonded to a heteroatom on the ring, wherein the ring is more preferably a pyrrole. In the case of a 6-membered ring, the amide or carboxylic acid (R₅) may be bonded to any ring atom to which R₁-R₄ is not bonded, but is desirably bonded to a carbon atom as exemplified in Formula II below.

In preferred aspects, the inventive compounds may have the structure illustrated below as Formulas I and II, wherein R₁-R₄ are as described herein.

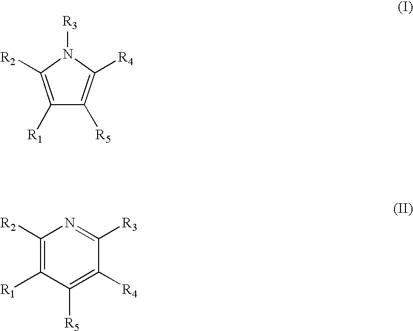

In the various aspects of the present invention, the first and second hydrophobic groups, R₂ and R₄, respectively, may be the same or different, and desirably comprise alkyls, alkenyls, alkynyls, heteroalkyls, cycloalkyls, heterocycloalkyls, acyls, aryls, heteroaryls, amino acids, or peptides comprising between 2 and 5 amino acids. More desirably, at least one of the hydrophobic groups (preferably R₂) is non-cyclic, and most desirably comprises alkyls, alkenyls and alkynyls, while the other hydrophobic group (preferably R₄) desirably comprises a cyclic moiety, and more desirably comprises an aryl, e.g., alkylaryl, alkenylaryl or alkynylaryl.

More desirably, the aforementioned hydrophobic groups comprise from 1 to 12, and more desirably 1 to 9 carbon atoms. Most desirably, one of the groups, desirably R₂, comprises from 1 to 6 carbon atoms, while the other group, desirably R₄, comprises from 3 to 12, or 3 to 9, carbon atoms.

While the carbon atoms in the hydrophobic groups may be linear or branched, it is preferred that at least one of the hydrophobic groups is branched. When a hydrophobic group is branched, it is desirable that the branched group (preferably R₂) comprise 4 to 6 carbon atoms. Preferably, this hydrophobic group comprises methylpropyl or methylpentyl, more preferably methylpentyl, and most preferably 2-methylpentyl.

The second hydrophobic group, desirably R₄, preferably comprises a ring, more desirably comprises an aryl, and even more preferably a phenyl, and even more desirably a halogen-substituted phenyl. More preferably the desired aryl group (e.g., phenyl) is linked to the ring atom via a C₁₋₄ alkyl, alkenyl or alkynyl, and more preferably by a C₂ alkyl, alkenyl or alkynyl. Most preferably, the second hydrophobic group comprises a halogen-substituted phenyl (e.g., chlorine, fluorine, etc.) which is linked to the ring structure via a C₂ linker, e.g., ethyl, ethenyl or enthynyl, with —(CH₂)₂(p-Cl-phenyl) being even more preferred.

In the various aspects of the present invention, the first and second moieties which each comprise a phosphate group, R₁ and R₃, respectively, may be the same or different, with the moiety comprising, in addition to the phosphate group, alkyls, alkenyls, alkynyls, heteroalkyls, cycloalkyls, heterocycloalkyls, acyls, aryls and heteroaryls. More desirably, the moieties comprise alkyls, alkenyls, alkynyls and aryls. Preferably, and in addition to the phosphate group, one of the moieties (preferably R₁) comprises a ring, desirably an aryl, while the other moiety (preferably R₃) comprises an alkyl, alkenyl or alkynyl.

R₁ comprises, more preferably, and in addition to the phosphate group, a 5- or 6-membered ring, and even more preferably an aryl, e.g., phenyl. Most preferably, R₁ comprises a substituted (desirably, halogen-substituted, e.g., chlorine, fluorine) phenyl group, and even more preferably chlorophenyl (e.g., 2-chlorophenyl phosphate).

R₃ comprises, more preferably and in addition to the phosphate group, an unsubstituted chain of 1-6 carbon atoms, even more preferably ethyl, ethenyl, ethynyl, propyl, propenyl or propynyl, and most preferably propyl, propenyl or propynyl.

R₅ may be an amide or carboxylic acid of any suitable structure, and desirably comprises —C₁₋₃(O)NH₂, —C₁₋₃(O)OH and more desirably comprises —C(O)NH₂ or —C(O)OH.

It is also contemplated that the preferred groups (R₁-R₅) disclosed herein may be used in various combinations. For example, the ring structure may desirably include R₂ and R₄, which may be the same or different, comprising alkyls, alkenyls, alkynyls, heteroalkyls, cycloalkyls, heterocycloalkyls, acyls, aryls, heteroaryls, amino acids, or peptides comprising between 2 and 5 amino acids, and R₁ and R₃, which may be the same or different, comprising alkyls, alkenyls, alkynyls, heteroalkyls, cycloalkyls, heterocycloalkyls, acyls, aryls or heteroaryls. More desirably, R₂ may be non-cyclic, R₄ may comprise a cyclic structure, R₁ may comprise an aryl, and R₃ may comprise an alkyl, alkenyl or alkynyl. Even more desirably, R₂ may comprise a C₁-C₁₂ alkyl, alkenyl or alkynyl, R₄ may comprise an aryl, R₁ may comprise a 5- or 6-membered aryl, and R₃ may comprise a C₁₋₆ alkyl, alkenyl or alkynyl. Preferably, R₂ may comprise a branched C₄-C₆ alkyl, alkenyl or alkynyl, R₄ may comprise an aryl which is linked to the ring by a C₁₋₄ alkyl, alkenyl or alkynyl, R₁ may comprise phenyl, and R₃ may comprise a C₁₋₃ alkyl, alkenyl or alkynyl, and more preferably wherein the ring comprises one nitrogen atom and the remaining ring atoms are carbon. Most preferably, R₂ may comprise methylpropyl or methylpentyl (even more preferably 2-methylpentyl, with the (S)-2-methylpentyl enantiomer being preferred relative to the (R)-2-methylpentyl enantiomer), R₄ may comprise phenyl linked to the ring via an ethyl group, R₁ may comprise a halogen-substituted phenyl, R₃ may comprise propyl, propenyl or propynyl, wherein R₅ comprises —C₁₋₃(O)NH₂ or —C₁₋₃(O)OH and even more preferably —C(O)NH₂ or —C(O)OH, and the ring is a single 5- or 6-membered ring, and more preferably a 5-membered ring (e.g., pyrrole). Prodrugs of each of the foregoing compounds are also contemplated by the invention.

Illustrative compounds contemplated by the present invention include those set forth in the following table (and prodrugs thereof). The table also provides information concerning the ability of each compound to inhibit phosphatase activity (Kᵢ(μM)).

TABLE 1

| Entry^{a} | Structure | Kᵢ(μM)^{b} |
|---|---|---|
| 1 | 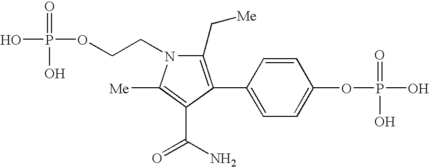 | NI |
| 2 | 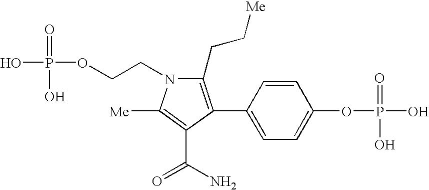 | NI |
| 3 | 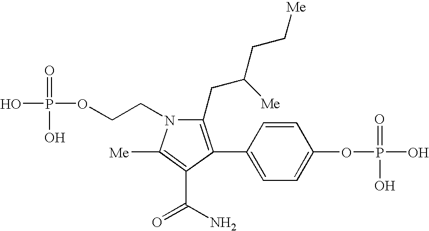 | 61 ± 15 |
| 4 | 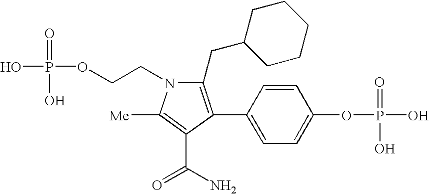 | 81 ± 5 |
| 5 | 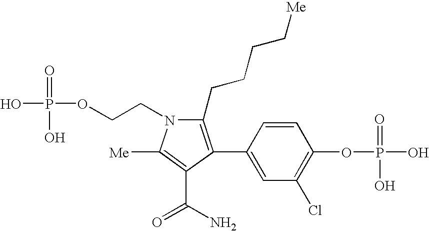 | 77 ± 12 |
| 6 | 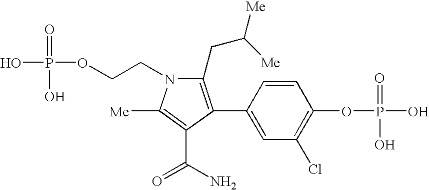 | 38 ± 8 |
| 7 | 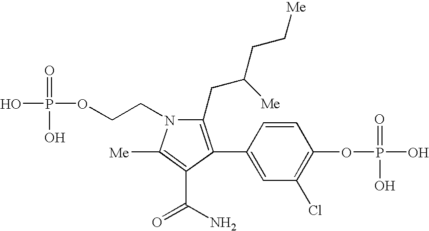 | 40 ± 1 |
| 8 | 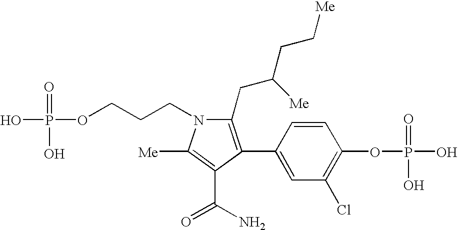 | 17 ± 1 |
| 9 | 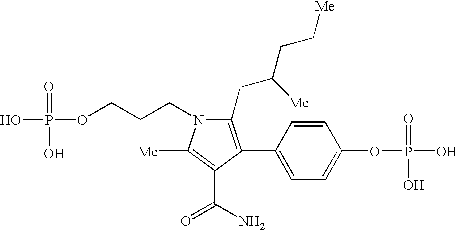 | 22 ± 3 |
| 10 | 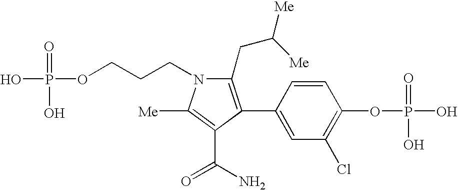 | 28 ± 1 |
| 11 | 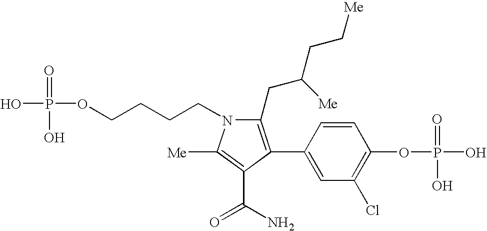 | NI |
| 12 | 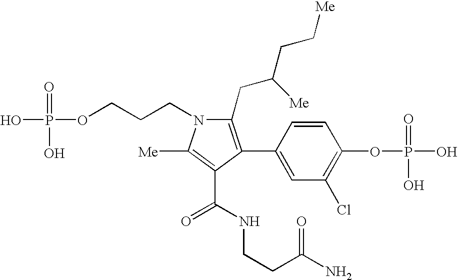 | NI |
| 13 | 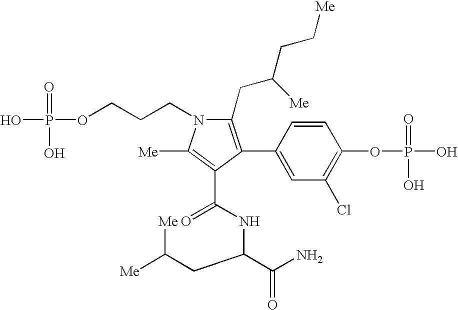 | 22 ± 2 |
| 14 | 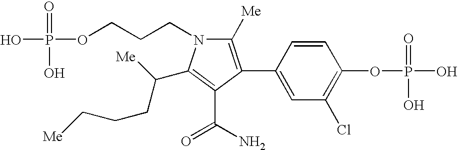 | NI |
| 15 | 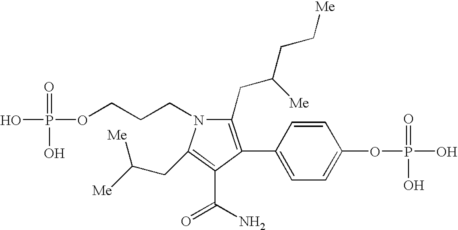 | 48 ± 5 |
| 16 | 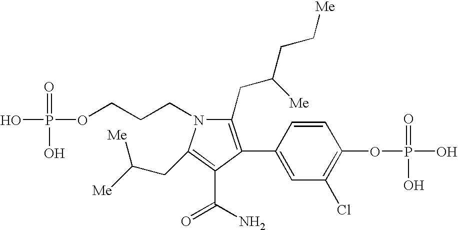 | 43 ± 3 |
| 17 | 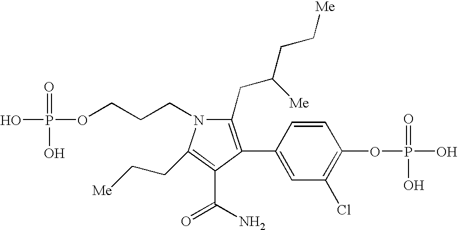 | 16 ± 1 |
| 18 | 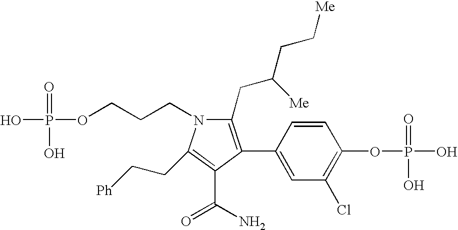 | 15 ± 1 |
| 19 | 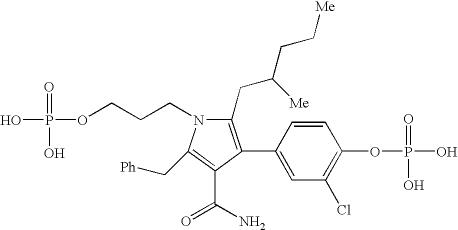 | 32 ± 4 |
| 20 | 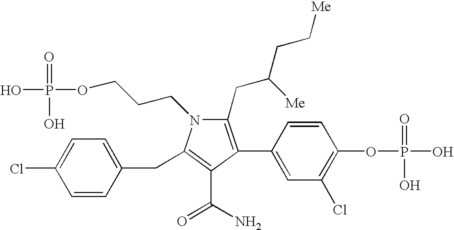 | 6.2 ± 0.6 |
| 21 | 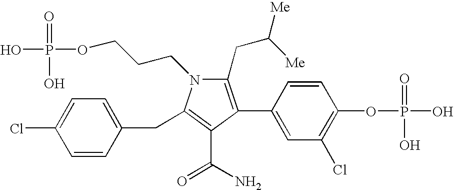 | 22 ± 1 |
| 22 | 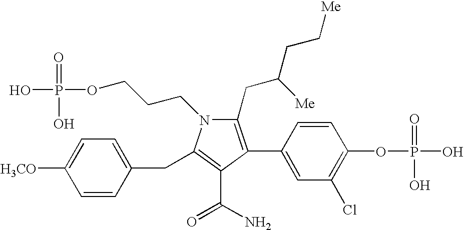 | 20 ± 2 |
| 23 | 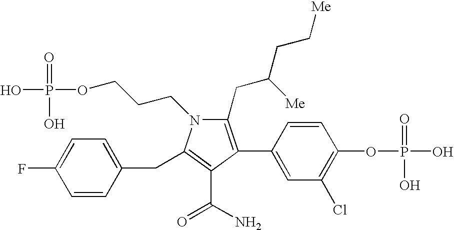 | 16 ± 2 |
| 24 | 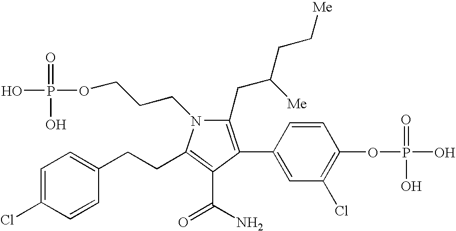 | 5.7 ± 0.4 |
| 25 | 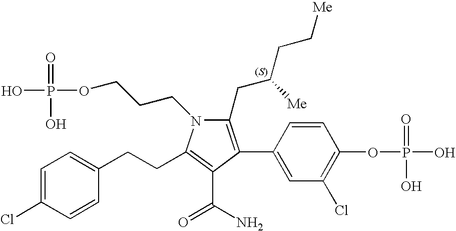 | 4.7 ± 0.7 |
| 26 | 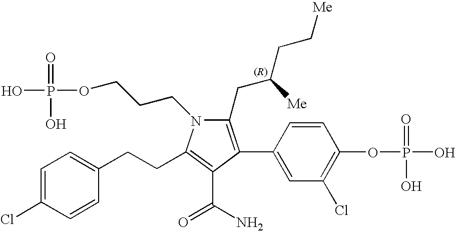 | 10 ± 1 |

The inhibition constant (Kᵢ) is used to determine the inhibitive effect of the inventive compounds on Wip1. A Kᵢ of about 10 μM or less is desirable in a Wip1 inhibiting compound. More preferably, a Kᵢ of less than about 5, even more preferably less than about 3 μM, even more preferably less than about 2, and most preferably less than about 1 μM, is desired. The Kᵢ was determined as described in the Example using the formula as set forth below

Kᵢ=IC₅₀/(1+[S]/Kₘ)

wherein [S] is the concentration of the substrate peptide and Kₘ is the Michaelis constant. A compound having a Kᵢ of less than about 5 μM was considered to be a Wip1 inhibitor. NI indicates that no Wip1 inhibition was observed.

The inventive compounds (which include prodrugs thereof), which may be referred to herein as Wip1 inhibitors, inhibit the biological activity of the Wip1 protein. These compounds, for example, block Wip1 from binding its substrate, alter the subcellular localization of Wip1, promote Wip1 degradation, and/or inhibit Wip1 phosphatase activity. Preferably, the compounds inhibit Wip1 phosphatase activity. One of ordinary skill in the art will appreciate that any degree of inhibition of Wip1 biological activity can produce a beneficial or therapeutic effect. As such, the invention does not require complete inhibition of Wip1 biological activity. Rather, varying degrees of inhibition are within the scope of the invention. In this respect, the compound preferably inhibits at least 10% of Wip1 biological activity. More preferably, a compound inhibits at least 50% of Wip1 biological activity, and most preferably 90% or more of Wip1 biological activity.

The phosphatase activity of the Wip1 protein in a cell can be inhibited to any level through the inventive method. Preferably, at least 10% (e.g., at least 20%, 30%, or 40%) of Wip1 phosphatase activity in a cell is inhibited upon administration of an inventive compound described herein. More preferably, at least 50% (e.g., at least 60%, 70% or 80%) of Wip1 phosphatase activity in a cell is inhibited upon administration of an inventive compound described herein. Most preferably, at least 90% (e.g., at least 95%, 99%, or 100%) of Wip1 phosphatase activity in a cell is inhibited upon administration of a compound described herein. Methods of testing the inhibition of Wip1 phosphatase activity are known in the art and include phosphatase assays described in, for example, Yamaguchi et al., Biochemistry, 44: 5285-5294 (2005), Harder et al., Biochem J., 298: 395-401 (1994), and Bonella-Deana et al., Methods Enzymol., 366: 3-17 (2003).

It is furthermore preferred that a compound that inhibits Wip1 phosphatase activity is specific for Wip1, i.e., inhibits the biological activity of Wip1 as opposed to that of another phosphatase, such as protein phosphatase 2C-alpha (PP2Cα) or a K238D mutant of Wip1. A compound that specifically inhibits the biological activity of Wip1 may inhibit the biological activity of another phosphatase, but to a significantly lesser extent than the extent to which the compound inhibits Wip1 biological activity. Methods for determining the specificity of a Wip1 inhibitor are known in the art and are described herein in the Examples.

The inventive compounds described herein may be synthesized using any suitable method known in the art. Illustrative methods are provided herein.

The inventive method of inhibiting Wip1 activity in a cell comprises contacting a cell with at least one of the inventive compounds described herein. The cell may be contacted with one, 2 or more, 5 or more compounds of the invention concurrently or in sequence. That is, a cell may be contacted with one or more compounds at the same time or may be contacted with one compound and then subsequently contacted with another of the inventive compounds.

The cell may be any suitable cell in which the compound can be introduced and stably maintained. The cell may be a eukaryotic cell or a prokaryotic cell (e.g., a bacteria cell), but is preferably a eukaryotic cell. Eukaryotic cells include cells of yeast, fungi, plants, algae, birds, reptiles, and mammals. When the cell is a eukaryotic cell, the cell is preferably a mammalian cell. In this regard, the cell can be isolated or derived from any suitable tissue or organ system. The cell may be a cell that replicates indefinitely in culture (i.e., a “transformed cell”), or the cell can be a primary cell that does not replicate indefinitely in culture. When the cell is a mammalian cell, it is preferably a human cell.

The compound may contact the cell in vitro. As used herein, the term “in vitro” means that the cell to which the compound is being administered is not within a living organism. Alternatively and preferably, the compound may be administered to the cell in vivo. As used herein, the term “in vivo” means that the cell is a part of a living organism. The compound may be administered to a host, e.g., a mammal, ex vivo, wherein the compound is administered to cells in vitro, and the cells are subsequently administered to the host.

In a preferred embodiment of the invention, the cell is a human cancer cell. The cancer can comprise a solid tumor or a tumor associated with soft tissue (i.e., soft tissue sarcoma) in a human. The cell can be associated with cancers of (i.e., located in) the oral cavity and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma or neuroblastoma), or the endocrine system (e.g., thyroid) and is not necessarily a cell of a primary tumor. Tissues associated with the oral cavity include, but are not limited to, the tongue and tissues of the mouth. Cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, non-small cell lung carcinoma. Tumors can arise in the uterine cervix, uterine corpus, ovary, vulva, vagina, prostate, testis, and penis, which make up the male and female genital systems, and the urinary bladder, kidney, renal pelvis, and ureter, which comprise the urinary system. The target tissue also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). Preferably, the cancer is breast cancer, colon cancer, neuroblastoma, adenocarcinoma, or ovarian cancer.

The inventive method of inhibiting Wip1 activity in a cell desirably is used to treat cancer in a human. As used herein, the term “treat” does not necessarily imply complete elimination of a cancer or inhibition of metastasis. Rather, there are varying degrees of treatment of which one of ordinary skill in the art recognizes as having a benefit or therapeutic effect. In this respect, the cancer can be treated to any extent through the present inventive method. For example, at least 10% (e.g., at least 20%, 30%, or 40%) of the growth of a cancerous tumor desirably is inhibited upon administration of a compound described herein. Preferably, at least 50% (e.g., at least 60%, 70%, or 80%) of the growth of a cancerous tumor is inhibited upon administration of a compound described herein. More preferably, at least 90% (e.g., at least 95%, 99%, or 100%) of the growth of a cancerous tumor is inhibited upon administration of a compound described herein. In addition or alternatively, the inventive method may be used to inhibit metastasis of a cancer.

The compound that inhibits Wip1 may be a part of a composition, such as a pharmaceutical composition, also referred to as a formulation. In this regard, the invention provides a composition comprising an inventive compound, preferably prodrugs as described herein, and a carrier, such as a pharmaceutically acceptable carrier. More than one compound (preferably, prodrugs) may be present in the composition. For example, 2 or more, or 5 or more, of the inventive compounds may be present in a given composition. Any suitable pharmaceutically acceptable carrier may be used within the context of the invention, and such carriers are well known in the art. The choice of carrier will be determined, in part, by the particular site to which the composition is to be administered and the particular method used to administer the composition.

Suitable compositions include aqueous and non-aqueous solutions, isotonic sterile solutions, which can contain anti-oxidants, buffers, bacteriostats, and solutes that render the composition isotonic with the blood or other bodily fluid of the intended recipient, and aqueous and non-aqueous sterile suspensions that can include suspending agents, solubilizers, thickening agents, stabilizers, and preservatives. Preferably, the pharmaceutically acceptable carrier is a liquid that contains a buffer and a salt. The composition may be presented in unit-dose or multi-dose sealed containers, such as ampules and vials, and may be stored in a freeze-dried (lyophilized) condition requiring only the addition of the sterile liquid carrier, for example, water, immediately prior to use. Extemporaneous solutions and suspensions may be prepared from sterile powders, granules, and tablets. Preferably, the pharmaceutically acceptable carrier is a buffered saline solution.

The choice of carrier will be determined in part by the particular compound employed in the composition, as well as by the particular method used to administer the composition. The following compositions for topical, oral, aerosol, parenteral, subcutaneous, intravenous, intramuscular, intraperitoneal, rectal, and vaginal administration are exemplary and are in no way limiting. One skilled in the art will appreciate that these administration routes are known. Although more than one route may be used to administer a particular composition, a particular route can provide a more immediate and more effective response than another route. If, for example, the cell is part of a solid tumor, the composition preferably is administered peritumorally or intratumorally.

The inventive composition may be formulated for injection. Injectable formulations are well-known to those of ordinary skill in the art (see, e.g., Pharmaceutics and Pharmacy Practice, J.B. Lippincott Company, Philadelphia, Pa., Banker and Chalmers, eds., pages 238 250 (1982), and ASHP Handbook on Injectable Drugs, Toissel, 4th ed., pages 622 630 (1986)).

The composition may be formulated for topical administration. Topical formulations are well known to those of skill in the art. For example, a drug reservoir or monolithic matrix transdermal patch device can be used for such topical administration, as can creams, ointments, or salves.

The composition may be formulated for oral administration. Formulations suitable for oral administration include, for example, (a) liquid solutions comprising a compound described herein dissolved in diluents, such as water, saline, or dextrose solutions, (b) capsules, sachets, tablets, lozenges, and troches, each containing a predetermined amount of the compound, as solids or granules, (c) powders, (d) suspensions in an appropriate liquid, and (e) suitable emulsions. Liquid formulations may include diluents, such as water and alcohols, for example, ethanol, benzyl alcohol, and the polyethylene alcohols, either with or without the addition of a pharmaceutically-acceptable surfactant. Capsule forms may be of the ordinary hard or soft shelled gelatin type containing, for example, surfactants, lubricants, and inert fillers, such as lactose, sucrose, calcium phosphate, and corn starch. Tablet forms may include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms may comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, excipients known in the art.

The compounds described herein, alone, in combination with another Wip1 inhibitor (such as a cyclic-phosphopeptide), or in combination with other suitable components, may also be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

The composition may be formulated for parenteral administration. Formulations suitable for parenteral administration include aqueous and non-aqueous isotonic sterile injection solutions, which can contain anti-oxidants, buffers, bacteriostats, and solutes that render the formulation isotonic with the blood of the intended recipient, and aqueous and non-aqueous sterile suspensions that may include suspending agents, solubilizers, thickening agents, stabilizers, and preservatives. The compounds described herein may be formulated for parenteral administration in combination with a carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically-acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils which may be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts. Suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates and 2-alkyl-imidazoline quaternary ammonium salts, and (e) mixtures thereof.

Ideally, the parenteral formulations will typically contain from about 0.5% to about 25% by weight of a particular compound in solution. The parenteral formulations may also contain preservatives and buffers. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. Extemporaneous injection solutions and suspensions can be prepared from sterile powders, granules, and tablets of the kind previously described.

Additionally, the compounds described herein can be made into suppositories by mixing with a variety of bases, such as emulsifying bases or water-soluble bases. Formulations suitable for vaginal administration can be presented as pessaries, tampons, creams, gels, pastes, foams, or spray formulas.

In addition, the composition may comprise additional therapeutic or biologically-active agents. For example, therapeutic factors useful in the treatment of a particular indication can be present. Factors that control inflammation, such as ibuprofen or steroids, may be part of the composition to reduce swelling and inflammation associated with in vivo administration of the composition and physiological distress. Immune system suppressors may be administered with the composition to reduce any immune response to the composition itself or associated with a disorder. Alternatively, immune enhancers can be included in the composition to upregulate the body's natural defenses against disease (e.g., cancer). Moreover, cytokines can be administered with the composition to attract immune effector cells to the tumor site.

One of ordinary skill in the art will readily appreciate that the compounds described herein can be modified in any number of ways, such that the therapeutic efficacy of the inhibitor is increased through the modification. For example, a compound may be conjugated either directly or indirectly through a linker to a targeting moiety. The practice of conjugating inhibitors to targeting moieties is known in the art (see, e.g., Wadwa et al., J. Drug Targeting, 3: 111 (1995), and U.S. Pat. No. 5,087,616). The term “targeting moiety” as used herein refers to any molecule or agent that specifically recognizes and binds to a cell-surface receptor, such that the targeting moiety directs the delivery of the compound to a population of cells on which surface the receptor is expressed. Targeting moieties include, but are not limited to, antibodies, or fragments thereof, peptides, hormones, growth factors, cytokines, and any other naturally- or non-naturally-existing ligands, which bind to cell surface receptors. The term “linker” as used in this context, refers to any agent or molecule that connects the compound to the targeting moiety. One of ordinary skill in the art will recognize that the attachment of the linker and targeting moiety to the compound should be such that they do not adversely and significantly interfere with the desired function of the compound, i.e., the ability to inhibit Wip1 activity in a cell.

The prodrugs contemplated herein are preferred when it is desired to administer the compounds disclosed herein to a mammal, e.g., as part of a therapeutic regimen for a condition or disease such as cancer. The term “prodrug” as used herein refers to any compound that when administered to a biological system generates a biologically-active compound as a result of spontaneous chemical reaction, enzyme catalyzed chemical reaction and/or metabolic chemical reaction, or a combination thereof.

The structure and preparation of prodrugs of the phosphate-containing compounds described herein will be appreciated by those skilled in the art. For example, prodrugs may be formed using groups attached to a phosphate, carboxylic acid or amine group. These groups are well known and include, by way of non-limiting illustration, alkyls, aryls, heteroaryls and the like. For example, when forming a prodrug from a carboxylic acid, an ester is provided. The term alkyl has the meaning generally understood by those skilled in the art and includes linear, branched, or cyclic alkyl moieties. C₁₋₆ alkyl esters are particularly useful, where the alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, and the like.

By way of further illustration, a review of phosphorus prodrugs is provided by Krise et al. Advanced Drug Delivery Reviews, 19, 287-310 (1996). Other examples of and information pertinent to prodrugs are provided in: H. Bundgaard ed., Design of Prodrugs (Elsevier 1985); K. Widder et al. eds., Methods in Enzymology, 42, 309-396 (Academic Press 1985); Krosgaard-Larsen and H, Bundgaard eds., A Textbook of Drug Design and Development (Ch. 5: “Design and Application of Prodrugs, 113-191) (1991); H. Bundgaard, Advanced Drug Delivery Reviews, 8, 1-38 (1992); H. Bundgaard et al., Journal of Pharmaceutical Sciences, 77, 285 (1988); and N. Kakeya et al., Chem. Phar. Bull., 32, 692 (1984).

For purposes of the inventive method, the amount or dose of the compound administered to a cell should be sufficient to effect the desired response, e.g., a therapeutic, response, over a reasonable time frame. The dose of the compound should be sufficient to inhibit Wip1 phosphatase activity in a cell within about 1-2 hours, if not 3-4 hours, from the time of administration. When the compound is administered to an animal in vivo, the dose of compound (preferably the prodrug thereof) will be determined by the efficacy of the particular compound and the condition of the animal (e.g., human), as well as the body weight of the animal (e.g., human). Many assays for determining a suitable dose of a compound are known in the art. For example, an assay which compares the extent to which the phosphatase activity of a Wip1 protein is inhibited in a cell upon administration of a given dose of a compound described herein to a mammal among a set of mammals that are each given a different dose of the compound could be used to determine a starting dose to be administered to an animal (e.g., a human). The extent to which the phosphatase activity of the Wip1 protein is inhibited upon administration of a certain dose of a compound can be assayed as described in the Examples and in Fiscella et al., supra.

The dose of compound also will be determined by the existence, nature, and extent of any adverse side effects that might accompany the administration of a particular compound. Ultimately, the attending physician will decide the dosage of the compound with which to treat each individual patient, taking into consideration a variety of factors, such as age, body weight, general health, diet, sex, inhibitor to be administered, route of administration, and the severity of the condition being treated.

The following examples further illustrate the invention but, of course, should not be construed as in any way limiting its scope.

### EXAMPLES

The following experimental procedures were used in the Examples described herein.

### Protein Expression and Purification

An N-terminal histidine-tagged, catalytic domain of the human Wip1 protein (amino acid residues 1-420), rWip1, and K238D mutant of Wip1 were expressed in Escherichia coli BL21 (DE3) and purified as previously reported in Yamaguchi et al., supra. The PP2A catalytic subunit and PP2Cα were purchased from Promega (Madison, Wis.) and Calbiochem (La Jolla, Calif.), respectively.

### Phosphatase Assay

Phosphatase activity was measured by a malachite green/molybdate-based assay (see, e.g., Yamaguchi et al., Biochemistry, 44:5285-5294 (2005), Harder et al, Biochem J., 298: 395-401 (1994), and Donella-Deana et al., Methods Enzymol., 366: 3-17 (2003)). The IC₅₀ values for inhibition of phosphatase activity by the inhibitors were measured using 30 μM AFEEGpSQSTTI substrate peptide (residues 1976-1986 in human ATM kinase) for 7 min at 30° C. in 50 mM Tris-HCl, pH 7.5, 0.1 mM EGTA, 0.02% 2-mercaptoethanol, 40 mM NaCl, and 30 mM MgCl₂. The inhibitors were pre-equilibrated at 30° C. for 6 min. The inhibition percentages were estimated by the following equation.

Inhibition (%)=100[1−(A−A₀)/(A₁₀₀−A₀)]

where A and A₁₀₀ denote absorbance intensities at 650 nm with and without the inhibitor, respectively. A₀ denotes absorbance of the sample without phosphatase. The IC₅₀ values were estimated by a sigmoidal dose-response equation. The apparent inhibitory constant (Kᵢ) values were estimated using the following equation (see, e.g., Cheng et al., Biochem. Pharmacol., 22: 3099-3108 (1973)):

Kᵢ=IC₅₀/(1+[S]/Kₘ)

wherein [S] is the concentration of the substrate peptide and Kₘ is the Michaelis constant.

### Steady-State Kinetics Assay

Kinetics assays were carried out in the manner described above. The amount of phosphate released was calculated using a phosphate standard curve. To determine the kinetic parameters Kₘ (the dissociation constant) and k_{cat} (first order rate constant), the initial velocities (v) were measured at various concentrations of substrate peptide ([S]), and data were fitted to the Michaelis-Menten equation, which is set forth below.

v=k_{cat}[S]/(Kₘ+[S])

For inhibition experiments, the initial velocities were measured at various concentrations of substrate peptide with a constant concentration of inhibitor ([I]). Lineweaver-Burke plots were used to assess the type of inhibition. The inhibition constant (Kᵢₛ) value was obtained by fitting the data to the competitive inhibition equation set forth below.

v=k_{cat}[S]/(Kₘ(1+[I]/Kᵢₛ)+[S])

### Computational Methods

Molecular modeling of the Wip1/inhibitor complex was performed using the atomic-scale, computer model of the active site of Wip1 as previously described (Yamaguchi et al, supra, Yamaguchi et al, Biochemistry, 45, 13193-13202 (2006)). This was a homology model developed from the crystal structure of PP2Cα. Topology files and initial coordinates of the different pyrrole-based inhibitors were made with the 2-D Sketcher and 3-D Builder modules of the QUANTA-2006 molecular modeling program (Accelrys, San Diego, Calif.). Energy minimization calculations were then done with the CHARMM (c31b2) molecular mechanics software package (Brooks et al., J. Comp. Chem., 4:187-217 (1983)) using the “par_a1122_prot” parameter set (MacKerrel at al., J. Phys. Chem. B., 102: 3586-3613 (1998)).

Examination of the range of energetically favorable interactions of the inhibitors with Wip1 was done with the integrated Autodock3 (Morris et al., J. Comp. Chem., 19: 1639-1662 (1998)) and AutodockTools (Sammer, J. Mol. Graphics. Modell., 17: 57-61 (1999)) docking software. In these simulations, all single bonds of the inhibitors were allowed to rotate freely. The protocol of the docking runs was the same as previously used (see Yamaguchi 2006, supra). Rotations and translations were done according to Lamarckian genetic algorithm, with a population size of 150 and a maximum number of generations of 1500. Each inhibitor was tested with at least 440 independent runs, with randomly selected dihedral angles and starting positions. The grid was a cube of 33.75 Å length (0.375 Å/point resolution), centered arbitrarily over the active site of Wip1.

### Example 1

This example demonstrates the synthesis of the compounds in accordance with preferred aspects of the invention.

Compounds with groups that mimic the phosphotyrosine, phosphoserine, isoleucine, and valine residues of phosphopeptide Wip1 inhibitor c(MpSIpYVA) were produced on a new scaffold. To make the compounds, a synthetic route based on the work by Jung and coworkers (Tetrahedron Lett., 39: 8263-8266 (1998)) was developed (see Scheme 1). Initially, β-ketoamides were synthesized on solid support by the combination of Rink amide resin with acylated derivatives of Meldrum's acid. Next, addition of an amine to form an enaminone on solid support, followed by addition of an α,β-unsaturated nitroalkene resulted in pyrrole formation. Deprotection, followed by phosphorylation and cleavage from the resin afforded the targeted pyrroles.

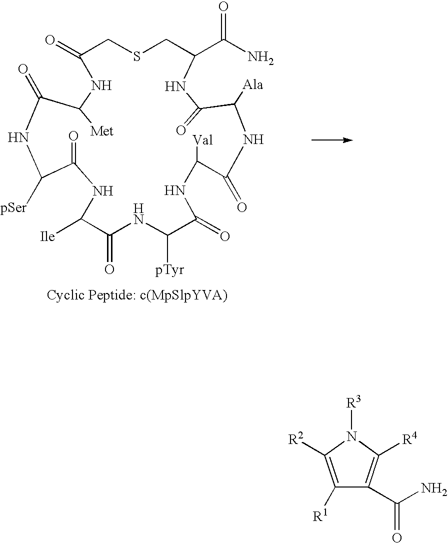

(A) Chemical structure of c(MpSIpYVA), (B) pyrrole scaffold to mimic the cyclic peptide.

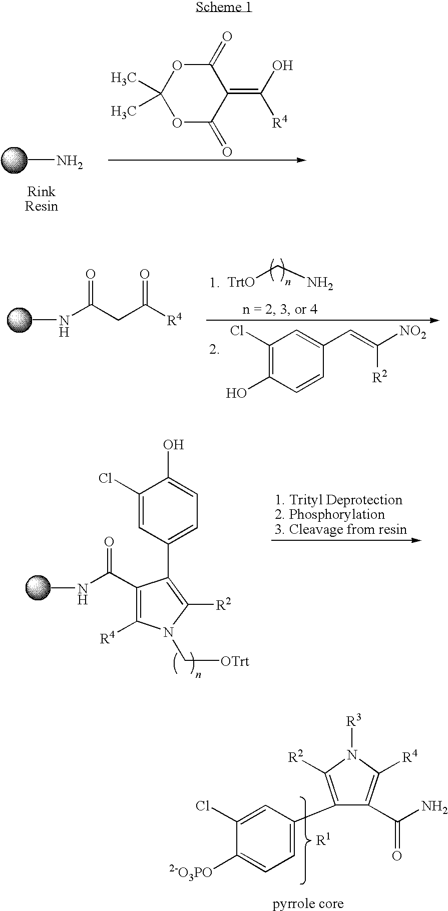

Solvents were reagent grade and dried prior to use. THF was distilled under N₂ from sodium/benzophenone immediately before use. All reactions were carried out under an argon atmosphere using dry solvents unless otherwise stated. Rink Amide resin was purchased from Novabiochem. (S)-(−)-1-amino-2-(methoxymethyl)pyrrolidine (SAMP) was purchased from ACROS. Unless otherwise noted, all other reagents and solvents were purchased from Aldrich and used without further purification. Analytical thin-layer chromatography (TLC) was carried out on Whatman TLC plates precoated with silica gel 60 (250 μm layer thickness). Visualization of the plates was accomplished using either a UV lamp, iodine and/or ninhydrin stain followed by heating. Flash chromatography was performed on EM Science silica gel 60 (230-400 mesh). Solvent mixtures used for TLC and column chromatography are reported in v/v ratios. Optical rotation values were measured on a Perkin-Elmer polarimeter. ¹H NMR spectra and ¹³C NMR spectra were recorded at 300 MHz and 75 MHz, respectively, on a variant GEMINI-300 spectrometer, using CDCl₃, CD₃OD or D₂O as solvent. Chemical shifts were reported in parts per million (ppm, δ) relative to tetramethylsilane (δ0.00). ³¹P NMR spectra were recorded using a Variant XL-300 spectrometer (121 Hz), orthophosphoric acid (85%) was used as an external standard. HPLC was carried out on a reversed-phase column, which was eluted with CH₃CN in 0.05% aqueous TFA and detected at OD 220 nm. Abbreviations used herein include: Dichloromethane, DCM; benzyl chloroformate, Cbz-Cl; N,N-diisopropylethylamine, DIEA; ethyl acetate, EA; and trityl chloride, Trt-Cl;

### Synthesis of 1° Amine Derivatives

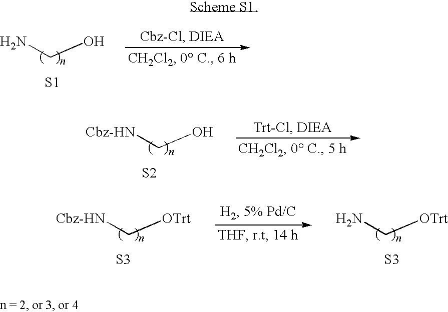

Cbz-aminopropanol (S2, n=3): To a cooled solution (0° C.) of 3-amino-1-propanol (S1) (2 g, 26.6 mmol, 1.0 equiv.) in DCM (30 mL) were slowly added Cbz-Cl (3.6 mL, 31.9 mmol, 1.2 equiv.) and DIEA (2.9 mL, 31.9 mmol, 1.2 equiv.). The mixture was allowed to warm to room temperature over 6 h before quenching with aqueous 5% AcOH (20 mL). The aqueous phase was extracted with DCM (2×20 mL), the combined organic extracts washed with aqueous NaHCO₃ (20 mL) and brine (30 mL), dried (MgSO₄) and concentrated in vacuo. Purification by flash chromatography (silica gel; EA-hexane, 3:1, R_{f} 0.37) gave 3.8 g (68%) of S2 as a white solid. ¹H NMR: (300 MHz, CDCl₃) δ 7.37-7.31 (m, 5H), 5.11 (s, 2H), 5.08 (br s, 1H), 3.44-3.41 (m, 2H), 3.36-3.31 (m, 2H), 2.54 (br s, 1H), 1.74-1.66 (m, 2H); ¹³C NMR: (75 MHz, CDCl₃) δ 157.46, 136.59, 128.65, 128.27, 128.19, 66.93, 59.67, 37.93, 32.56; ESI-MS, m/z 210.1 for [M+H]⁺ (calcd for C₁₁H₁₆N₂O₃ 210.2).

N-Cbz-O-trityl-propanolamine (S3, n=3): To a cooled solution (0° C.) of S2 (2 g, 9.6 mmol, 1.0 equiv.) in DCM (30 mL) were slowly added Trt-Cl (3.2 g, 11.5 mmol, 1.2 equiv.) and DIEA (2.0 mL, 11.5 mmol, 1.2 equiv.). The mixture was allowed to warm to room temperature over 5 h before being quenched with aqueous 5% AcOH (20 mL). The aqueous phase was extracted with DCM (2×20 mL), the combined organic extracts washed with aqueous NaHCO₃ (20 mL) and brine (30 mL), dried (MgSO₄) and concentrated in vacuo. Purification by flash chromatography (silica gel; EA-hexane, 1:7, R_{f} 0.29) gave 3.2 g (75%) of S3 as a white solid. ¹H NMR: (300 MHz, CDCl₃) δ 7.43-7.21 (m, 20H), 5.07 (s, 2H), 3.33-3.30 (m, 2H), 3.20-3.16 (m, 2H), 1.81-1.77 (m, 2H); ¹³C NMR: (75 MHz, CDCl₃) 156.50, 144.21, 128.76, 128.64, 128.16, 128.11, 128.09, 128.03, 127.41, 127.20, 66.63, 62.02, 39.42, 29.92; ESI-MS, m/z 452.1 for [M+H]⁺ (calcd for C₃₀H₃₀N₂O₃ 452.2).

O-trityl-propanolamine (S4, n=3): S3 (2 g, 4.4 mmol) was added to a 100 mL RBF followed by THF (30 mL). 5% Pd/C (0.4 g) was added to the reaction mixture, then hydrogen was introduced to the solution by a gas inlet tube with stirring for 14 h. The reaction mixture was filtered, and concentrated on a rotary evaporator to give 1.3 g (92%) of S4 as a colorless oil. R_{f} 0.26 (CHCl₃-MeOH, 7:1); ¹H NMR: (300 MHz, CDCl₃) δ 7.45-7.22 (m, 15H), 3.15 (t, J=6.0 Hz, 2H), 2.85 (t, J=6.9 Hz, 2H), 1.90 (br s, 2H), 1.82-1.75 (m, 2H); ¹³C NMR: (75 MHz, CDCl₃) δ 144.21, 128.76, 128.65, 128.16, 128.11, 128.09, 128.03, 127.41, 127.20, 66.63, 62.02, 39.42, 29.92; ESI-MS, m/z 318.2 for [M+H]⁺ (calcd for C₂₂H₂₄NO 318.2).

Other compounds of S4 (where n=2 or 4) were made using the same procedures started from ethanolamino or 4-amino-1-butanol, respectively.

### Synthesis of Nitroalkenes

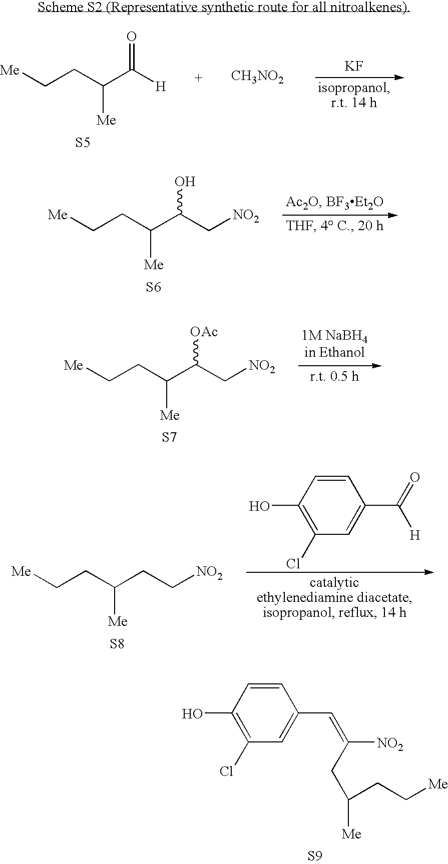

All nitroalkenes synthesized using the route in Scheme S2.

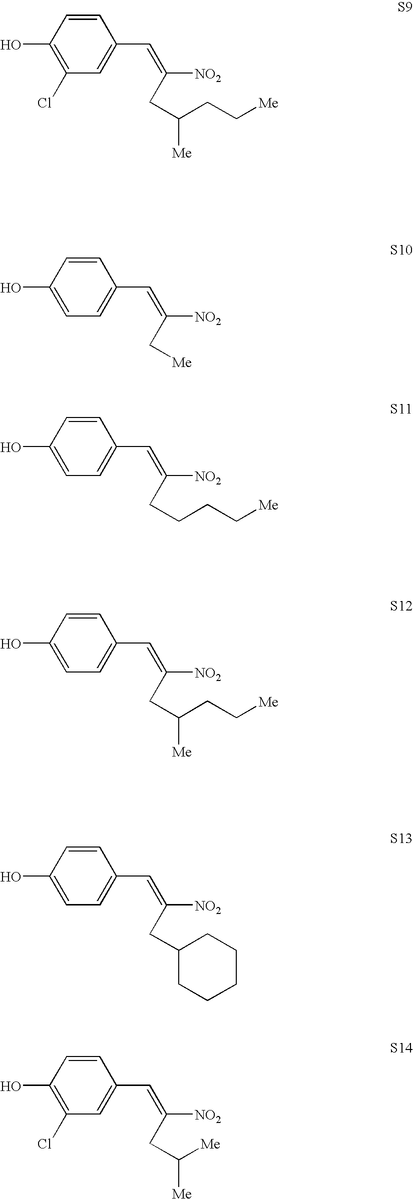

3-methyl-1-nitro-hexan-2-ol (S6): To a solution of 2-methylpentanal (S5) (3.9 g, 39 mmol, 1.0 equiv.) in isopropanol (30 mL) were added potassium fluoride (KF, 0.22 g, 3.9 mmol, 0.1 equiv.) and nitromethane (4.7 mL, 78 mmol, 2 equiv.). The mixture was stirred at room temperature for 14 h before being quenched with aqueous 5% AcOH (20 mL). The aqueous phase was extracted with EA (2×20 mL), the combined organic extracts were washed with aqueous NaHCO₃ (20 mL) and brine (30 mL), dried (MgSO₄) and concentrated in vacuo. Purification by flash chromatography (silica gel; EA-hexane, 1:5, R_{f} 0.41) gave 3.9 g (62%) of S6 as a colorless oil. Mixture of diastereomers ¹H NMR: (300 MHz, CDCl₃) δ 4.46-4.42 (m, 2H), 4.29-4.16 (m, 1H), 1.81-1.55 (m, 1H), 1.51-1.39 (m, 4H), 0.96-0.90 (m, 6H); ¹³C NMR: (75 MHz, CDCl₃) δ 79.27, 79.18, 72.87, 72.09, 36.88, 36.50, 35.08, 34.39, 20.32, 20.21, 15.10, 14.33, 14.29, 14.22; ESI-MS, m/z 162.2 for [M+H]⁺ (calcd for C₇H₁₆NO₃ 162.1).

Acetic acid-2-methyl-1-nitromethyl-pentylester (S7): To a cooled solution (0° C.) of 3-methyl-1-nitro-hexan-2-ol (S6) (1.6 g, 9.8 mmol, 1.0 equiv.) in THF (30 mL) were added acetic anhydride (3.7 mL, 49 mmol, 5 equiv.) and boron trifluoride-diethyl etherate (BF₃.Et₂O, 0.5 mL, 4.9 mmol, 0.5 equiv.). The mixture was stirred for 20 h at 4° C. before being quenched with aqueous NaHCO₃ (20 mL). The aqueous phase was extracted with EA (2×50 mL), the combined organic extracts were washed with brine (30 mL), dried (MgSO₄) and concentrated in vacuo. Purification by flash chromatography (silica gel; EA-hexane, 1:10, R_{f} 0.33) gave 1.7 g (85%) of S7 as a colorless oil. Mixture of diastereomers ¹H NMR: (300 MHz, CDCl₃) δ 5.52-5.40 (m, 1H), 4.64-4.50 (m, 2H), 2.07 (s, 3H), 1.91-1.83 (m, 1H), 1.61-1.21 (m, 4H), 0.97-0.90 (m, 6H); ¹³C NMR: (75 MHz, CDCl₃) δ 170.02, 147.92, 138.67, 76.24, 75.63, 73.76, 73.25, 34.95, 34.77, 34.55, 34.53, 20.78, 20.37, 20.19, 19.87, 14.71, 14.15, 14.00; ESI-MS, m/z 204.2 for [M+H]⁺ (calcd for C₉H₁₈NO₄ 204.1).

3-methyl-1-nitro-hexane (S8): 1 M ethanolic sodium borohydride (20 mL) was added to S7 (2.0 g, 9.4 mmol) with stirring. After 0.5 h, the mixture was acidified with hydrochloric acid (1 M, 20 mL) then extracted with EA (2×50 mL). The organic extracts were washed with brine (20 mL), dried (MgSO₄) and concentrated in vacuo. Purification by flash chromatography (silica gel; EA-hexane, 1:10, R_{f} 0.68) gave 1.2 g (87%) of S8 as a colorless oil. ¹H NMR: (300 MHz, CDCl₃) δ 4.41 (t, J=7.2 Hz, 1H), 2.11-2.01 (m, 1H), 1.87-1.77 (m, 1H), 1.57-1.51 (m, 1H), 1.39-1.15 (m, 4H), 0.96-0.90 (m, 6H); ¹³C NMR: 8 (75 MHz, CDCl₃) 74.36, 38.90, 34.52, 30.24, 20.04, 19.25, 14.31; ESI-MS, m/z 146.1 for [M+H]⁺ (calcd for C₇H₁₆NO₃, 146.1).

2-chloro-4-(4-methyl-[E]-2-nitro-hep-1-enyl)-phenol (S9): To a solution of 3-chloro-4-hydroxy-benzaldehyde (2 g, 12.8 mmol, 1.0 equiv.) in isopropanol (20 mL) were added S8 (5.6 g, 38.4 mmol, 3.0 equiv.) and ethylenediamine diacetate (0.35 g, 1.92 mmol, 0.15 equiv). The mixture was refluxed for 14 h. The reaction mixture was diluted with EA (50 mL) and the organic layer washed with aqueous 5% AcOH (2×20 mL), brine (30 mL), dried (MgSO₄) and concentrated in vacuo. Purification by flash chromatography (silica gel; EA-hexane, 1:3, R_{f} 0.44) gave 1.26 g (35%) of S9 as a yellow oil. ¹H NMR: (300 MHz, CDCl₃) δ 7.93 (s, 1H), 7.47 (d, J=2.1 Hz, 1H), 7.33-7.30 (m, 1H), 7.10 (d, J=8.4 Hz, 1H), 2.93-2.86 (m, 1H), 2.78-2.70 (m, 1H), 1.86-1.80 (m, 1H), 1.40-1.11 (m, 4H), 0.90-0.85 (m, 6H); ¹³C NMR: (75 MHz, CDCl₃) δ 153.09, 151.44, 132.68, 130.84, 130.57, 125.93, 120.83, 117.05, 39.30, 34.00, 32.02, 20.21, 19.49, 14.00; ESI-MS, m/z 284.1 for [M+H]⁺ (calcd for C₁₄H₁₉ClNO₃ 284.1).

The nitroalkenes shown in Scheme 2 (S10-S14) were synthesized by the same synthetic route shown in Scheme S2 using the appropriate aldehydes. The characterization data for these compounds is shown below.

S10: ¹H NMR: (300 MHz, CDCl₃) δ 7.99 (s, 1H), 7.40 (d, J=7.8 Hz, 2H), 6.94 (d, J=7.8 Hz, 2H), 5.30 (s, 1H), 2.90 (q, J=7.5 Hz, 2H), 1.28 (t, J=7.2 Hz, 3H); ¹³C NMR: (75 MHz, CDCl₃) δ 154.04, 150.68, 132.63, 129.81, 128.99, 124.93, 120.20, 117.02, 34.45, 31.28; ESI-MS, m/z 192.1 for [M−H]⁻ calcd for C₁₀H₁₀NO₃ 192.1).

S11: ¹H NMR: (300 MHz, CDCl₃) δ 7.91 (s, 1H), 7.43 (d, J=1.8 Hz, 1H), 7.29-7.26 (m, 2H), 7.12 (d, J=8.7 Hz, 1H), 5.81 (br, 1H), 2.84-2.79 (m, 2H), 1.64-1.61 (m, 2H), 1.41-1.37 (m, 4H), 0.92 (t, J=6.6 Hz, 3H); ¹³C NMR: (75 MHz, CDCl₃) δ 154.09, 151.44, 132.23, 130.71, 130.33, 125.93, 120.20, 117.02, 31.62, 34.00, 32.02, 20.21, 19.49, 14.00; ESI-MS, m/z 268.1 for [M−H]⁻ (calcd for C₁₃H₁₅ClNO₃ 268.1).

S12: ¹H NMR: (300 MHz, CDCl₃) δ 8.02 (s, 1H), 7.47 (d, J=7.8 Hz, 2H), 6.91 (d, J=8.7 Hz, 2H), 2.96-2.88 (m, 1H), 2.82-2.72 (m, 1H), 1.87-1.80 (m, 1H), 1.40-1.12 (m, 4H), 0.89-0.85 (m, 6H); ¹³C NMR: (75 MHz, CDCl₃) δ 153.09, 151.44, 134.20, 132.30, 116.29, 39.39, 34.17, 32.06, 20.32, 19.55, 14.67; ESI-MS, m/z 248.1 for [M+H]⁺ (calcd for C₁₄H₁₈NO₃ 248.1).

S13: ¹H NMR: (300 MHz, CDCl₃) δ 8.02 (s, 1H), 7.41 (d, J=8.7 Hz, 2H), 6.92 (d, J=8.7 Hz, 2H), 2.85 (d, J=7.5 Hz, 2H), 1.72-1.61 (m, 5H), 1.37-1.12 (m, 4H), 1.11-0.98 (m, 2H); ¹³C NMR: (75 MHz, CDCl₃) δ 157.63, 149.85, 134.31, 132.31, 125.00, 116.27, 37.14, 34.26, 33.26, 26.34; ESI-MS, m/z 260.1 for [M−H]⁻ (calcd for C₁₅H₁₈ClNO₃ 260.1).

S14: ¹H NMR: (300 MHz, CDCl₃) δ 7.94 (s, 1H), 7.47 (d, J=2.1 Hz, 1H), 7.33-7.30 (m, 1H), 7.10 (d, J=8.7 Hz, 1H), 2.82 (d, J=7.2 Hz, 2H), 2.06-1.94 (m, 1H), 0.96 (d, J=6.6 Hz, 6H); ¹³C NMR: (75 MHz, CDCl₃) δ 152.95, 151.43, 132.64, 130.83, 130.56, 126.03, 120.79, 117.05, 35.40, 27.75, 22.50; ESI-MS, m/z 254.0 for [M−H](calcd for C₁₂H₁₄ClNO₃ 254.1).

### Asymmetric Synthesis of Chiral Nitroalkenes

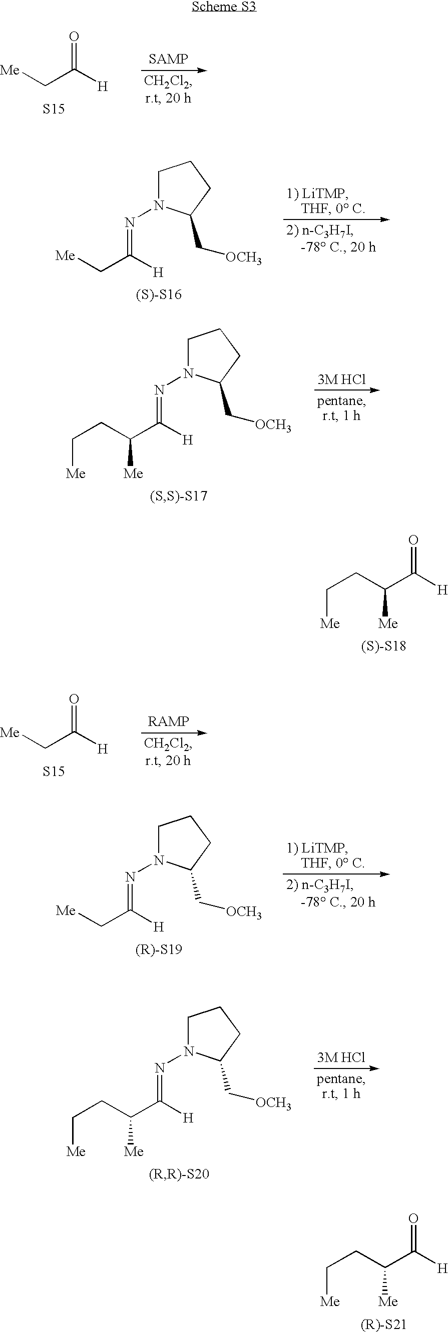

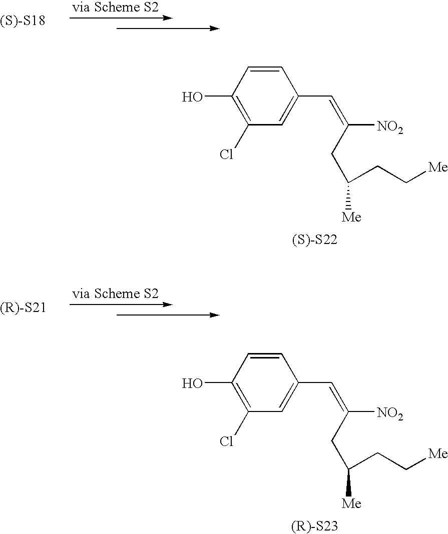

(S)-(−)-2-Methoxymethyl-1-(1′-propylidenamino)-pyrrolidine, [(S)-S16]: To a cooled solution (0° C.) of (S)-(−)-1-amino-2-(methoxymethyl)-pyrrolidine (SAMP, 2.0 g, 15.4 mmol, 1.0 equiv.) in DCM (15 mL), 4 Å molecular sieves (1 g) and propanal (S15) (1.3 mL, 18.5 mmol, 1.2 equiv.) were added sequentially. The mixture was stirred at room temperature for 20 h. The reaction mixture was diluted with DCM (30 mL) and filtered. The filtrate was dried (MgSO₄) and concentrated in vacuo to give a pale yellow oil. Purification by flash chromatography (silica gel; pentane-Et₂O, 4:1, containing 1% Et₃N, R_{f} 0.48) gave 2.6 g (95%) of (S)-S16 as a colorless oil. [α]_{D}²⁰ −132.9° (c 1.65, C₆H₆), lit.³ [α]_{D}²² −146°; ¹H NMR: (300 MHz, CDCl₃) δ 6.61 (t, J=5.4 Hz, 1H), 3.58-3.57 (m, 1H), 3.40-3.37 (m, 3H), 3.38 (s, 3H), 2.73-2.70 (m, 1H), 2.28-2.19 (m, 2H), 1.95-1.87 (m, 4H), 1.06 (t, J=7.5 Hz, 3H); ¹³C NMR: (75 MHz, CDCl₃) δ 140.60, 75.01, 63.65, 59.33, 50.58, 26.74, 26.56, 22.29, 12.26; ESI-MS, m/z 171.1 for [M+H]⁺ (calcd for C₉H₁₉N₂O 171.2).

(2S,2′S)-2-Methoxymethyl-1-(2′-methyl-1′-pentyliden-amino)pyrrolidine, [(S,S)-S17]: To a cooled solution (0° C.) of 2,2,6,6-tetramethylpiperidine (3.1 mL, 18.2 mmol, 1.2 equiv.) in dry THF (20 mL) under Ar was slowly added n-BuLi [10.0 mL, 18.1 mmol, 1.81 M, 1.2 equiv (calculated from titration)]; the mixture was stirred for 1 h. A solution of (S)-S16 (2.6 g, 15.2 mmol, 1.0 equiv.) in dry THF (5 mL) was added slowly and stirring maintained at 0° C. for 1 h. The resulting orange solution was cooled to −78° C. and 1-iodopropane (1.8 mL, 18.2 mmol, 1.2 equiv.) added dropwise. The mixture was allowed to warm to room temperature over 20 h before being quenched with pH 7 buffer (10 mL). The aqueous phase was extracted with Et₂O (2×20 mL), the combined organic extracts washed with aqueous NH₄Cl (30 mL) and brine (30 mL), dried (MgSO₄) and concentrated in vacuo. Purification by flash chromatography (silica gel; pentane-Et₂O, 4:1, containing 1% Et₃N, R_{f} 0.61) gave 1.8 g (56%) of (S,S)-S17 as a colorless oil. [α]_{D}²⁰ −116.4 (c 1.0, C₆H₆), lit.³ [α]_{D}²² −124°; ¹H NMR: (300 MHz, CDCl₃) δ 6.53 (d, J=6.6 Hz, 1H), 3.60-3.56 (m, 1H), 3.45-3.33 (m, 3H), 3.38 (s, 3H), 2.73-2.68 (m, 1H), 2.34-2.30 (m, 1H), 1.95-1.77 (m, 4H), 1.42-1.29 (m, 4H), 1.04 (d, J=6.9 Hz, 3H), 0.92 (t, J=6.6 Hz, 3H); ¹³C NMR: (75 MHz, CDCl₃) δ 145.06, 75.04, 63.79, 59.39, 50.82, 38.02, 37.10, 26.76, 22.31, 20.54, 19.19, 14.41; ESI-MS, m/z 213.1 for [M+H]⁺ (calcd for C₁₂H₂₅N₂O 213.2).

(S)-(−)-2-Methylpentanal, [(S)-S18]: A solution of the hydrazone (S,S)-S17 (1.5 g, 7.0 mmol) in pentane (20 mL) was stirred with aqueous 3 M HCl (10 mL) for 1 h. The two phases were separated, and the aqueous phase was extracted with Et₂O (2×20 mL). The combined organic extracts were washed with aqueous NaHCO₃ (20 mL) and brine (30 mL), dried (MgSO₄) and used in the subsequent nitroalkene synthesis steps shown in Scheme S2 without additional purification.

Using the same procedures for the synthesis of (S)-S18, S15 was converted to (R)-S21 using (R)-(+)-1-amino-2-(methoxy-methyl)pyrrolidine (RAMP) as the chiral auxiliary.

(S)-2-chloro-4-(4-methyl-[E]-2-nitro-hep-1-enyl)-phenol [(S)-S22] and (R)-2-chloro-4-(4-methyl-[E]-2-nitro-hep-1-enyl)-phenol [(R)-S23]: In the same manner as described in Scheme S2 for the synthesis of nitroalkene S9, (S)-S18 and (R)-S21 were converted to (S)-S22 and (R)-S23 (28%, 30%), respectively [All data (¹H, ¹³C and ESI-mass) were identical with those of compound S9].

### Synthesis of Meldum's Acid Derivatives

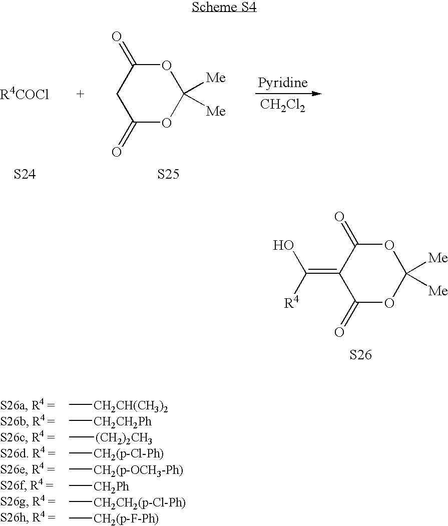

The general procedure used was acylation of Meldrum's acid. To a solution of 2,2-dimethyl-1,3-dioxane-4,6-dione (S25) (Meldrum's acid, 2.0 g, 13.9 mmol, 1.0 equiv.) in DCM (20 mL) pyridine (1.5 mL, 27.8 mmol, 2.0 equiv.) was added. The mixture was stirred at 25° C. for 1 h. The resulting red solution was cooled to 0° C. (ice bath) and an acid chloride S24 (in this example, 4-chlorophenylacetyl chloride (1.9 mL, 15.3 mmol, 1.1 equiv.)) was added dropwise. The mixture was allowed to warm to room temperature over 20 h before being quenched with 1 M HCl (15 mL). The aqueous phase was extracted with DCM (3×15 mL), the combined organic extracts were dried (MgSO₄) and concentrated in vacuo. The crude product was used for the next step without further purification (purity in all cases was >90% based on ¹H NMR).

### Characterization Data for All Derivatives of Meldrum's Acid

S26a: ¹H NMR: (300 MHz, CDCl₃) δ 3.00 (d, J=6.6 Hz, 2H), 2.26-2.06 (m, 1H), 1.74 (s, 6H), 1.03 (d, J=6.6 Hz, 6H); ¹³C NMR: (75 MHz, CDCl₃) δ 171.39, 161.54, 94.14, 42.95, 25.21, 22.43; ESI-MS, m/z 227.1 for [M−H] (calcd for C₁₁H₁₅O₅, 227.1).

S26b: ¹H NMR: (300 MHz, CDCl₃) δ 7.30-7.20 (m, 5H), 3.41 (t, J=7.5 Hz, 2H), 3.14 (t, J=7.8 Hz, 2H), 1.67 (s, 6H); ¹³C NMR: (75 MHz, CDCl₃) δ 171.00, 161.50, 139.75, 128.78, 128.38, 126.73, 106.64, 93.84, 32.00, 25.20; ESI-MS, m/z 275.1 for [M−H] (calcd for C₁₅H₁₅O₅, 275.1).

S26c: ¹H NMR: (300 MHz, CDCl₃) δ 3.06 (t, J=7.5 Hz, 2H), 1.82-1.78 (m, 2H), 1.67 (s, 6H), 1.03 (q, J=7.2 Hz, 3H); ¹³C NMR: (75 MHz, CDCl₃) δ 172.07, 161.61, 93.34, 35.65, 25.14, 19.29, 13.61; ESI-MS, m/z 213.1 for [M−H] (calcd for C₁₀H₁₃O₅, 213.1).

S26d: ¹H NMR: (300 MHz, CDCl₃) δ 7.32-7.26 (m, 4H), 4.38 (s, 2H), 1.72 (s, 6H); ¹³C NMR: (75 MHz, CDCl₃) δ 194.12, 164.05, 160.79, 131.17, 131.00, 129.06, 128.98, 105.33, 91.69, 40.33, 27.07; ESI-MS, m/z 297.3 for [M+H]⁺ (calcd for C₁₄H₁₄ClO₅, 297.3).

S26e: ¹H NMR: (300 MHz, CDCl₃) δ 7.33 (d, J=7.8 Hz, 2H), 6.87 (d, J=7.8 Hz, 2H), 4.36 (s, 2H), 3.79 (s, 3H), 1.74 (s, 6H); ¹³C NMR: (75 MHz, CDCl₃) δ 191.23, 132.25, 130.58, 114.38, 55.44, 50.29, 40.26, 29.29; ESI-MS, m/z 291.1 for [M−H] (calcd for C₁₅H₁₅O₆, 291.1).

S26f: ¹H NMR: (300 MHz, CDCl₃) δ 7.38-7.26 (m, 5H), 4.43 (s, 2H), 1.72 (s, 6H); ¹³C NMR: (75 MHz, CDCl₃) δ 194.52, 170.60, 160.31, 129.56, 128.66, 127.45, 104.92, 91.46, 50.85, 40.72, 26.71; ESI-MS, m/z 261.1 for [M+H]⁺ (calcd for C₁₄H₁₅O₅, 261.1).

S26g: ¹H NMR: (300 MHz, CDCl₃) δ 7.30-7.26 (m, 4H), 3.42 (t, J=7.5 Hz, 2H), 3.02 (t, J=7.8 Hz, 2H), 1.72 (s, 6H); ¹³C NMR: (75 MHz, CDCl₃) δ 196.57, 170.60, 160.31, 139.79, 128.70, 128.61, 126.64, 105.02, 91.98, 37.31, 32.09, 26.84; ESI-MS, m/z 310.2 for [M+H]⁺ (calcd for C₁₅H₁₆ClO₅, 310.1)

S26h: ¹H NMR: (300 MHz, CDCl₃) δ 7.40-7.35 (m, 2H), 7.04-6.98 (m, 2H), 4.38 (s, 2H), 1.72 (s, 6H); ¹³C NMR: (75 MHz, CDCl₃) δ 194.44, 164.05, 160.79, 131.46, 131.36, 115.79, 115.60, 105.25, 91.57, 40.11, 27.00; ESI-MS, m/z 280.2 for [M+H]⁺ (calcd for C₁₄H₁₄FO₅, 280.1).

### Solid Phase Synthesis of Compounds for Wip1 Inhibition

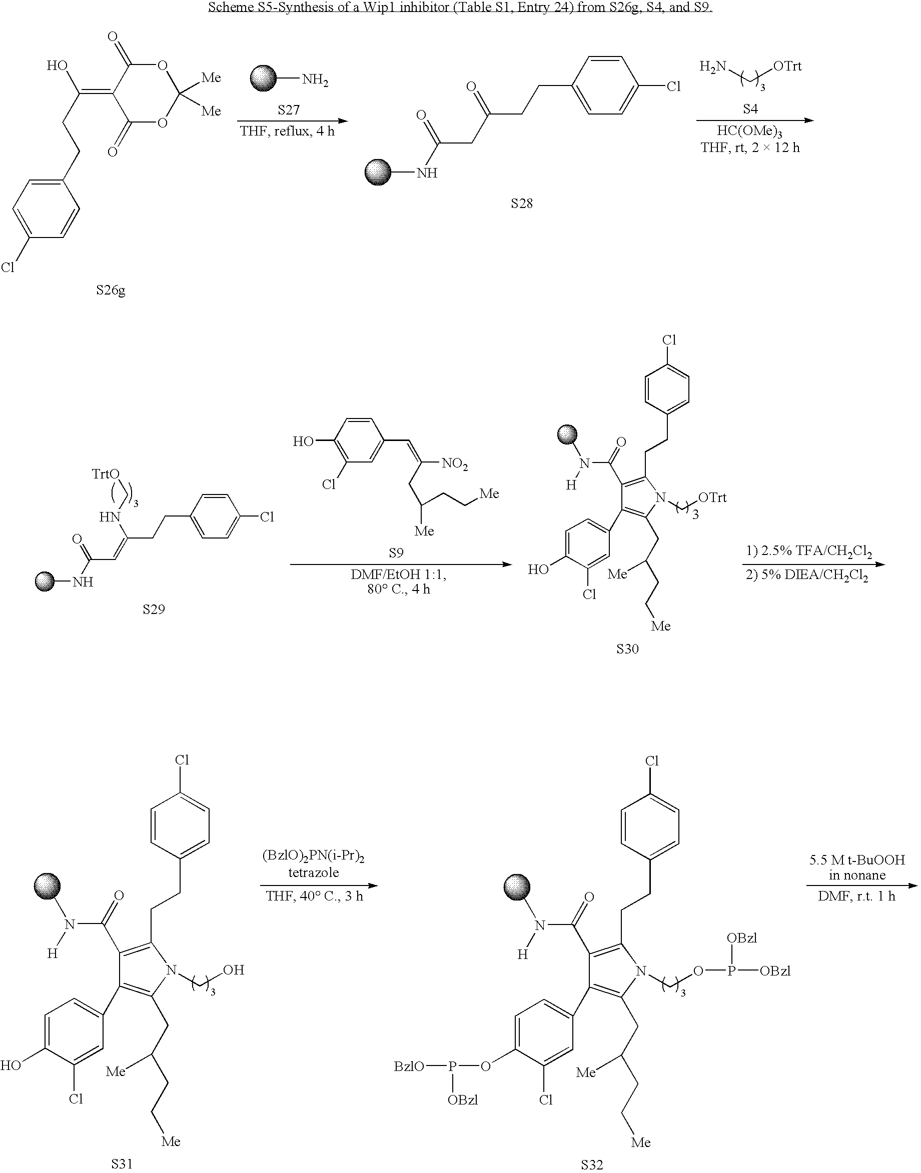

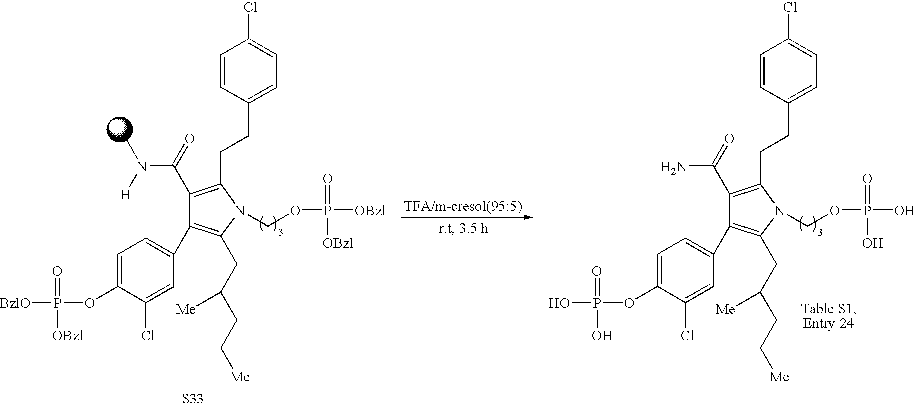

### General Procedure 2: Solid Phase Synthesis of Pyrroles

The following procedure uses the synthesis of the molecule from Table S1, Entry 24 as an example, using S26g, S4, and S9 as synthetic components for the synthesis. The sample synthesis is shown graphically in Scheme S5.

β-ketoamide resin S28: Rink amide resin S27 (0.5 g, capacity: 0.6 mmol/g) was suspended in DMF/piperidine 1:1 (5 mL) and shaken for 45 min. The resin was washed with DMF (2×10 mL), THF (2×10 mL) and this step was repeated. The Kaiser ninhydrin test gave a positive result (blue color). The resin was suspended in THF (10 mL) and an acylated Meldrum's acid (in this example S26g, 0.9 g, 3.0 mmol, 10 equiv.) was added. The reaction mixture was heated at reflux. After 4 h, the resin was washed with THF (3×5 mL), DCM (3×5 mL), Et₂O (3×5 mL) and dried under vacuum. The Kaiser ninhydrin test of S28 gave a negative result (colorless). This resin was used for the next step.

Enaminone resin S29: To a suspension of resin S28 (0.5 g, 0.3 mmol, 1.0 equiv.) in THF (3 mL) were added trimethylorthoformate (0.3 mL, 3 mmol, 10 equiv.) and a trityl-protected amino alcohol (in this case, O-trityl-propanolamine (S4) 0.9 g, 3 mmol, 10 equiv.) at 25° C. The reaction mixture was stirred for 12 h, then the resin was washed with THF (3×5 mL) and this step was repeated once more. The reaction mixture was washed successively with THF (3×5 mL), DCM (3×5 mL) and Et₂O (3×5 mL) and dried under high vacuum. This resin was used for the next step.

Pyrrole synthesis on the solid support S30:⁴ To a suspension of enaminone resin S29 (0.5 g, 0.3 mmol, 1.0 equiv.) in DMF/EtOH 1:1 (5 mL) was added a nitroalkene (in this case, 2-chloro-4-(4-methyl-[E]-2-nitro-hep-1-enyl)-phenol (S9) 0.43 g, 1.5 mmol, 5 equiv.). The reaction mixture was stirred at 80° C. for 4 h, after which the resin was filtered, washed successively with DMF (3×5 mL), DCM (3×5 mL) and Et₂O (3×5 mL) and dried under high vacuum.

When it was necessary to check this reaction, a small portion of the pyrrole was deprotected and cleaved from the resin. An aliquot of the resin (0.1 g, 0.06 mmol) was treated with TFA (4 mL) in the presence of triisopropylsilane (0.1 mL) at room temperature for 1 h. After evaporation of TFA, CHCl₃ (5 mL) was added to the reaction vessel. The organic layer was washed with aqueous NaHCO₃ (3 mL) and dried (MgSO₄). Purification of the crude product by preparative thin layer chromatography (silica gel CHCl₃-MeOH 9:1, R_{f} 0.33) gave, in this case, free S30 (minus the Trt-protecting group) as a colorless oil. ¹H NMR: (300 MHz, CDCl₃) δ 7.29-7.02 (m, 7H), 5.98 (br s, 1H), 5.02 (br s, 2H), 3.68-3.63 (m, 4H), 3.24 (t, J=7.5 Hz, 2H), 2.99 (t, J=7.5 Hz, 2H), 2.45 (dd, J=14.7, 6.6 Hz, 1H), 2.20 (dd, J=14.7, 8.4 Hz, 1H), 1.83-1.78 (m, 2H), 1.59 (br s, 1H), 1.12-1.08 (m, 3H), 0.94-0.89 (m, 1H), 0.79 (t, J=7.5 Hz, 3H), 0.65 (d, J=6.6 Hz, 3H); ¹³C NMR: (75 MHz, CDCl₃) δ 167.28, 141.53, 133.27, 131.33, 129.65, 128.48, 128.23, 128.03, 125.87, 120.50, 118.88, 115.47, 62.54, 38.32, 36.42, 33.06, 30.84, 19.36, 19.28, 14.04; ESI-MS, m/z 517.2 for [M+H]⁺ (calcd for C₂₈H₃₅Cl₂N₂O₃ 517.2).

Deprotection of Trt Group (S31): TFA (2.5%) in DCM (5 mL) was added to resin S30 (0.5 g, 0.3 mmol) and the mixture was agitated at 25° C. for 5 min. After filtration, the resin was re-treated with 2.5% TFA/DCM (5 mL) for 5 min. The resin was washed with DCM and DMF, then shaken with 5% DIEA/DCM for 30 s (three times). The resulting resin S31 was washed successively with DMF (3×5 mL), DCM (3×5 mL) and Et₂O (3×5 mL) and dried under high vacuum. This resin was used for phosphitylation step.

Phosphitylation (S32):⁵ In a flask was dissolved 1H-tetrazole (2.0 mL, 1.0 mmol, 10 equiv.) in DMF (2 mL) followed by dibenzyl-N,N-diisopropylphosphoramidite (0.2 mL, 1.0 mmol, 10 equiv.). After 5 min, the mixture was added to resin S31 (0.2 g, 0.1 mmol) in DMF (5 mL) and the mixture was stirred at 40° C. for 3 h. After this time, the resin was filtered on a sintered glass funnel, washed with DMF (3×5 mL). The resulting resin S32 was used for the next step immediately.

Oxidation (S33):⁵ To a stirred solution of resin S32 (0.2 g, 0.1 mmol) in DMF (3 mL) was added 5.5 M-t-butyl hydroperoxide in nonane (0.5 mL, 2.5 mmol, 25 equiv.) and the mixture was stirred at 25° C. for 1 h. After this time, the resulting resin S33 was washed successively with DMF (3×5 mL), DCM (3×5 mL) and Et₂O (3×5 mL) and dried under high vacuum.

Cleavage of pyrrole derivatives from resin: The resin S33 (0.2 g, 0.1 mmol) was treated with TFA/m-cresol (95/5=v:v) (5 mL) at 25° C. for 3.5 h. After evaporation of TFA, ether (15 mL) was added to the reaction vessel. The resulting precipitate was washed with ether (10 mL) and dissolved in 0.1% aqueous TFA. The solution was freeze-dried and the crude product was purified by preparative HPLC to give the final pyrrole as a white powder. All products were purified by reverse phase HPLC.

Purification and characterization data for compounds. Each compound was synthesized following General Procedure 2, and Scheme 5.

1: HPLC, 18.50 min [Agilent Eclipse XOB-C18 column (4.6×250 mm), 1.0 mL/min, CH₃CN (0% to 60%, 30 min)], ¹H NMR: (300 MHz, D₂O) δ 7.02 (d, J=8.4 Hz, 2H), 6.78 (d, J=6.9 Hz, 2H), 3.89 (t, J=6.6 Hz, 2H), 3.60-3.52 (br s, 2H), 2.49 (s, 3H), 2.38-2.36 (m, 2H), 0.97 (t, J=7.2 Hz, 3H); ³¹P NMR: (121 Hz, D₂O) δ 0.53, −3.92; ESI-MS, m/z 447.04 for [M−H]⁻ (calcd for C₁₆H₂₁N₂O₉P₂ 447.08).

2: HPLC, 19.87 min [Agilent Eclipse XOB-C18 column (4.6×250 mm), 1.0 mL/min, CH₃CN (0% to 60%, 30 min)], ¹H NMR: (300 MHz, D₂O) δ 7.06 (d, J=8.4 Hz, 2H), 6.76 (d, J=7.2 Hz, 2H), 3.86 (t, J=6.6 Hz, 2H), 3.58-3.50 (br s, 2H), 2.47 (s, 3H), 2.36-2.16 (m, 2H), 1.46-1.20 (m, 2H), 0.96 (t, J=7.2 Hz, 3H); ³¹P NMR: (121 Hz, D₂O) δ 0.53, −3.92; ESI-MS, m/z 461.09 for [M−H]⁻ (calcd for C₁₇H₂₄N₂O₉P₂ 461.10).

3: HPLC, 22.83 min [Agilent Eclipse XOB-C18 column (4.6×250 mm), 1.0 mL/min, CH₃CN (0% to 60%, 30 min)], ¹H NMR: (300 MHz, D₂O) δ 7.10-7.06 (m, 2H), 6.87-6.82 (m, 2H), 4.01 (t, J=7.8 Hz, 2H), 3.62 (t, J=7.8 Hz, 2H), 2.55-2.51 (m, 1H), 2.45 (s, 3H), 2.37-2.33 (m, 1H), 1.87-1.85 (m, 2H), 1.55-1.42 (m, 1H), 1.19-1.17 (m, 4H), 0.76 (t, J=7.2 Hz, 3H), 0.68 (d, J=6.6 Hz, 3H); ³¹P NMR: (121 Hz, D₂O) δ 0.54, −3.93; MALDI-TOF MS, m/z 517.15 for [M−H] (calcd for C₂₁H₃₁N₂O₉P₂ 517.16).

4: HPLC, 22.77 min [Agilent Eclipse XOB-C18 column (4.6×250 mm), 1.0 mL/min, CH₃CN (0% to 60%, 30 min)], ¹H NMR: (300 MHz, D₂O) δ 7.31-7.19 (m, 4H), 4.23 (t, J=5.7 Hz, 2H), 4.11-4.07 (m, 2H), 2.55 (d, J=7.2 Hz, 1H), 2.47 (s, 3H), 1.61-1.41 (m, 6H), 1.21-0.98 (m, 4H), 0.71-0.66 (m, 1H); ³¹P NMR: (121 Hz, D₂O) δ 0.50, −3.66; MALDI-TOF MS, m/z 515.11 for [M−H]⁻ (calcd for C₂₁H₂₉N₂O₉P₂ 515.12).

5: HPLC, 21.54 min [Agilent Eclipse XOB-C18 column (4.6×250 mm), 1.0 mL/min, CH₃CN (0% to 60%, 30 min)], ¹H NMR: (300 MHz, D₂O) δ 7.43-7.40 (m, 2H), 7.24-7.21 (m, 1H), 4.24 (t, J=6.0 Hz, 2H), 4.09 (t, J=7.8 Hz, 2H), 2.59 (t, J=8.1 Hz, 2H), 2.48 (s, 3H), 1.50-1.41 (m, 2H), 1.23-1.16 (m, 4H), 0.78-0.75 (m, 3H); ³¹P NMR: (121 Hz, D₂O) δ 0.54, −3.92; MALDI-TOF MS, m/z 523.00 for [M−H] (calcd for C₁₉H₂₆ClN₂O₉P₂ 523.03).

6: HPLC, 20.77 min [Agilent Eclipse XOB-C18 column (4.6×250 mm), 1.0 mL/min, CH₃CN (0% to 60%, 30 min)], ¹H NMR: (300 MHz, D₂O) δ 7.43-7.39 (m, 2H), 7.25-7.21 (m, 1H), 4.25 (t, J=7.2 Hz, 2H), 4.10 (t, J=6.6 Hz, 2H), 2.57 (d, J=7.5 Hz, 2H), 2.48 (s, 3H), 1.78-1.62 (m, 1H), 0.71 (d, J=6.6 Hz, 6H); ³¹P NMR: (121 Hz, D₂O) δ 0.53, −3.93; MALDI-TOF MS, m/z 509.10 for [M−H] (calcd for C₁₈H₂₅ClN₂O₉P₂ 509.07).

7: HPLC, 23.55 min [Agilent Eclipse XOB-C18 column (4.6×250 mm), 1.0 mL/min, CH₃CN (0% to 60%, 30 min)], ¹H NMR: (300 MHz, D₂O) δ 7.43-7.38 (m, 2H), 7.23-7.20 (m, 1H), 4.23 (t, J=5.7 Hz, 2H), 4.09 (t, J=6.3 Hz, 2H), 2.75-2.63 (m, 1H), 2.56-2.44 (m, 1H), 2.46 (s, 3H), 1.55-1.42 (m, 1H), 1.17-0.89 (m, 4H), 0.71 (t, J=7.2 Hz, 3H), 0.65 (d, J=6.6 Hz, 3H); ³¹P NMR: (121 Hz, D₂O) δ 0.54, −3.93; MALDI-TOF MS, m/z 537.10 for [M−H] (calcd for C₂₀H₂₈ClN₂O₉P₂ 537.10).

8: HPLC, 23.65 min [Agilent Eclipse XOB-C18 column (4.6×250 mm), 1.0 mL/min, CH₃CN (0% to 60%, 30 min)], ¹H NMR: (300 MHz, D₂O) δ 7.40-7.37 (m, 2H), 7.21-7.17 (m, 1H), 4.09-3.95 (m, 4H), 2.65-2.61 (m, 1H), 2.46-2.39 (m, 1H), 2.45 (s, 3H), 2.12-1.97 (m, 2H), 1.55-1.42 (m, 1H), 1.12-0.89 (m, 4H), 0.71 (t, J=6.9 Hz, 3H), 0.64 (d, J=6.6 Hz, 3H); ³¹P NMR: (121 Hz, D₂O) δ 0.54, −3.93; MALDI-TOF MS, m/z 551.13 for [M−H] (calcd for C₂₁H₃₀ClN₂O₉P₂ 551.12).

9: HPLC, 22.82 min [Agilent Eclipse XOB-C18 column (4.6×250 mm), 1.0 mL/min, CH₃CN (0% to 60%, 30 min)], ¹H NMR: (300 MHz, D₂O) δ 7.10-7.06 (m, 2H), 6.87-6.82 (m, 2H), 4.04 (t, J=7.8 Hz, 2H), 3.63 (t, J=6.0 Hz, 2H), 2.55-2.51 (m, 1H), 2.45 (s, 3H), 2.39-2.27 (m, 1H), 1.87-1.81 (m, 2H), 1.55-1.42 (m, 1H), 1.12-0.89 (m, 4H), 0.76 (t, J=6.6 Hz, 3H), 0.68 (d, J=6.6 Hz, 3H); ³¹P NMR: (121 Hz, D₂O) δ 0.53, −3.93; MALDI-TOF MS, m/z 517.14 for [M−H] (calcd for C₂₁H₃₁N₂O₉P₂ 517.16).

10: HPLC, 17.81 min [Agilent Eclipse XOB-C18 column (4.6×250 mm), 1.0 mL/min, CH₃CN (0% to 60%, 30 min)], ¹H NMR: (300 MHz, D₂O) δ 7.41-7.39 (m, 2H), 7.23-7.20 (m, 1H), 4.08 (t, J=8.1 Hz, 2H), 4.01-3.95 (m, 2H), 2.54 (d, J=8.1 Hz, 2H), 2.47 (s, 3H), 2.08-1.98 (m, 2H), 1.72-1.65 (m, 1H), 0.71 (d, J=6.6 Hz, 6H); ³¹P NMR: (121 Hz, D₂O) δ 0.53, −3.93; MALDI-TOF MS, m/z 523.23 for [M−H]⁻ (calcd for C₁₉H₂₆ClN₂O₉P₂ 523.09).

11: HPLC, 27.70 min [Agilent Eclipse XOB-C18 column (4.6×250 mm), 1.0 mL/min, CH₃CN (0% to 60%, 30 min)], ¹H NMR: (300 MHz, D₂O) δ 7.43-7.39 (m, 2H), 7.23-7.20 (m, 1H), 4.09-3.75 (m, 4H), 2.68-2.61 (m, 1H), 2.46-2.41 (m, 1H), 2.47 (s, 3H), 1.82-1.72 (m, 4H), 1.52-1.48 (m, 1H), 1.12-0.89 (m, 4H), 0.74 (t, J=6.9 Hz, 3H), 0.69 (d, J=6.6 Hz, 3H); ³¹P NMR: (121 Hz, D₂O) δ 0.54, −3.92; MALDI-TOF MS, m/z 565.12 for [M−H]⁻ (calcd for C₂₂H₃₂ClN₂O₉P₂ 565.13).

12: HPLC, 21.05 min [Agilent Eclipse XOB-C18 column (4.6×250 mm), 1.0 mL/min, CH₃CN (10% to 70%, 30 min)], ¹H NMR: (300 MHz, D₂O) δ 7.44-7.29 (m, 2H), 7.16-7.12 (m, 1H), 4.08-3.4.01 (m, 2H), 3.98-3.96 (m, 2H), 3.40 (t, J=6.3 Hz, 2H), 2.65-2.57 (m, 1H), 2.46-2.38 (m, 1H), 2.40 (s, 3H), 2.32 (t, J=6.0 Hz, 2H), 2.08-1.80 (m, 2H), 1.56-1.46 (m, 1H), 1.16-0.89 (m, 4H), 0.70 (t, J=6.6 Hz, 3H), 0.64 (d, J=6.6 Hz, 3H); ³¹P NMR: (121 Hz, D₂O) δ 0.54, −3.93; MALDI-TOF MS, m/z 622.20 for [M−H] (calcd for C₂₄H₃₅ClN₃O₁₀P₂ 622.16).

13: HPLC, 23.80 min [Agilent Eclipse XOB-C18 column (4.6×250 mm), 1.0 mL/min, CH₃CN (10% to 70%, 30 min)], ¹H NMR: (300 MHz, D₂O) δ 7.10-7.03 (m, 2H), 6.88-6.86 (m, 1H), 4.01-3.96 (m, 1H), 3.76-3.71 (m, 2H), 3.67-3.60 (m, 2H), 2.38-2.26 (m, 1H), 2.18-2.08 (m, 1H), 2.08 (s, 3H), 1.69-1.62 (m, 2H), 1.26-1.12 (m, 3H), 0.84-0.72 (m, 4H), 0.50-0.36 m, 12H); ³¹P NMR: (121 Hz, D₂O) δ 0.53, −3.92; MALDI-TOF MS, m/z 665.20 for [M] (calcd for C₂₇H₄₂ClN₃O₁₀P₂ 665.20).

14: HPLC, 20.73 min [Agilent Eclipse XOB-C18 column (4.6×250 mm), 1.0 mL/min, CH₃CN (0% to 60%, 30 min)], ¹H NMR: (300 MHz, D₂O) δ 7.12-7.01 (m, 2H), 7.03-7.00 (m, 1H), 4.03 (t, J=5.7 Hz, 2H), 4.77 (t, J=6.0 Hz, 2H), 2.11 (s, 3H), 1.92-1.90 (m, 1H), 1.82-1.78 (m, 2H), 1.61-0.58 (m, 4H), 1.42 (d, J=7.2 Hz, 3H), 0.90 (t, J=7.5 Hz, 3H); ³¹P NMR: (121 Hz, D₂O) δ 0.54, −3.92; MALDI-TOF MS, m/z 537.18 for [M−H] (calcd for C₂₀H₂₈ClN₂O₉P₂ 537.10).

15: HPLC, 17.48 min [Agilent Eclipse XOB-C18 column (4.6×250 mm), 1.0 mL/min, CH₃CN (10% to 70%, 30 min)], ¹H NMR: (300 MHz, D₂O) δ 7.31 (d, J=7.8 Hz, 2H), 7.23 (d, J=7.8 Hz, 2H), 4.26 (t, J=8.1 Hz, 2H), 4.03 (t, J=6.6 Hz, 2H), 2.83 (d, J=7.2 Hz, 2H), 2.63-2.58 (m, 1H), 2.50-2.41 (m, 1H), 1.92-1.87 (m, 1H), 1.61-1.56 (m, 1H), 1.20-1.09 (m, 4H), 0.92 (d, J=6.9 Hz, 6H), 0.75-0.67 (m, 6H); ³¹P NMR: (121 Hz, D₂O) δ 0.69, −3.90; MALDI-TOF MS, m/z 545.18 for [M−H]⁻ (calcd for C₂₃H₃₅N₂O₉P₂ 545.19).

16: HPLC, 18.01 min [Agilent Eclipse XOB-C18 column (4.6×250 mm), 1.0 mL/min, CH₃CN (10% to 70%, 30 min)], ¹H NMR: (300 MHz, D₂O) δ 7.41-7.38 (m, 2H), 7.23-7.19 (m, 1H), 4.15-4.06 (m, 2H), 3.98-3.94 (m, 2H), 2.83 (d, J=7.2 Hz, 2H), 2.63-2.58 (m, 1H), 2.50-2.41 (m, 1H), 2.59-1.95 (m, 2H), 1.92-1.87 (m, 1H), 1.61-1.56 (m, 1H), 1.32 (d, J=6.6 Hz, 2H), 1.20-1.09 (m, 2H), 0.92 (d, J=6.9 Hz, 6H), 0.75-0.67 (m, 6H); ³¹P NMR: (121 Hz, D₂O) δ 0.70, −3.91; MALDI-TOF MS, m/z 593.14 for [M−H]⁻ (calcd for C₂₄H₃₆ClN₂O₉P₂ 593.13).

17: HPLC, 25.27 min [Agilent Eclipse XOB-C18 column (4.6×250 mm), 1.0 mL/min, CH₃CN (10% to 70%, 30 min)], ¹H NMR: (300 MHz, D₂O) δ 7.41-7.38 (m, 2H), 7.24-7.20 (m, 1H), 4.16-4.07 (m, 2H), 3.98-3.96 (m, 2H), 2.87 (t, J=7.5 Hz, 2H), 2.68-2.61 (m, 1H), 2.51-2.43 (m, 1H), 2.21-2.30 (m, 2H), 1.61-1.56 (m, 3H), 1.96-1.14 (m, 4H), 0.96 (t, J=7.5 Hz, 3H), 0.75-0.66 (m, 6H); ³¹P NMR: (121 Hz, D₂O) δ 0.68, −3.91; MALDI-TOF MS, m/z 579.13 for [M−H]⁻ (calcd for C₂₃H₃₄ClN₂O₉P₂ 579.15).

18: HPLC: 23.61 min [Agilent Eclipse XOB-C18 column (4.6×250 mm), 1.0 mL/min, CH₃CN (10% to 70%, 30 min)]; ¹H NMR: (300 MHz, D₂O) δ 7.48-7.11 (m, 8H), 3.88-3.82 (m, 2H), 3.76-3.72 (m, 2H), 3.20 (t, J=7.2 Hz, 2H), 2.97 (t, J=6.6 Hz, 2H), 2.56-2.50 (m, 1H), 2.41-2.33 (m, 1H), 1.89-1.82 (m, 2H), 1.51-1.44 (m, 1H), 1.19-1.06 (m, 3H), 0.95-0.89 (m, 1H), 0.76 (t, J=7.2 Hz, 3H), 0.68 (d, J=6.9 Hz, 3H); ³¹P NMR: (121 Hz, D₂O) δ 0.69, −3.90; MALDI-TOF MS, m/z 641.41 for [M−H]⁻ (calcd for C₂₈H₃₆ClN₂O₉P₂ 641.17).

19: HPLC, 25.01 min [Agilent Eclipse XOB-C18 column (4.6×250 mm), 1.0 mL/min, CH₃CN (10% to 70%, 30 min)], ¹H NMR: (300 MHz, D₂O) δ 7.47-7.20 (m, 8H), 4.38 (s, 2H), 3.94 (t, J=7.5 Hz, 2H), 3.83 (t, J=5.4 Hz, 2H), 2.67-2.60 (m, 1H), 2.51-2.44 (m, 1H), 1.87-1.81 (m, 2H), 1.54-1.45 (m, 1H), 1.11-1.01 (m, 3H), 0.95-0.91 (m, 1H), 0.71 (t, J=6.9 Hz, 3H), 0.67 (d, J=6.6 Hz, 3H); ³¹P NMR: (121 Hz, D₂O) δ 0.69, −3.91; MALDI-TOF MS, m/z 627.58 for [M−H](calcd for C₂₇H₃₄ClN₂O₉P₂ 627.15).

20: HPLC, 26.72 min [Agilent Eclipse XOB-C18 column (4.6×250 mm), 1.0 mL/min, CH₃CN (10% to 70%, 30 min)], ¹H NMR: (300 MHz, D₂O) δ 7.47-7.14 (m, 7H), 4.34 (s, 2H), 3.94 (t, J=6.9 Hz, 2H), 3.87-3.81 (m, 2H), 2.67-2.60 (m, 1H), 2.51-2.44 (m, 1H), 1.89-1.74 (m, 2H), 1.54-1.45 (m, 1H), 1.11-1.01 (m, 3H), 0.95-0.91 (m, 1H), 0.71 (t, J=6.6 Hz, 3H), 0.66 (d, J=6.6 Hz, 3H); ³¹P NMR: (121 Hz, D₂O) δ 0.70, −3.91; MALDI-TOF MS, m/z 662.45 for [M−H]⁻ (calcd for C₂₇H₃₃C₁₂N₂O₉P₂ 662.42).

21: HPLC, 20.32 min [Agilent Eclipse XOB-C18 column (4.6×250 mm), 1.0 mL/min, CH₃CN (10% to 70%, 30 min)], ¹H NMR: (300 MHz, D₂O) δ 7.46-7.13 (m, 7H), 4.34 (s, 2H), 3.94 (t, J=6.9 Hz, 2H), 3.87-3.83 (m, 2H), 2.54 (d, J=7.5 Hz, 2H), 1.86-1.74 (m, 2H), 1.68-1.60 (m, 1H), 0.69 (d, J=6.6 Hz, 6H); ³¹P NMR: (121 Hz, D₂O) δ 0.71, −3.90; MALDI-TOF MS, m/z 633.45 for [M−H]⁻ (calcd for C₂₅H₂₉Cl₂N₂O₉P₂ 633.37).

22: HPLC, 29.80 min [Agilent Eclipse XOB-C18 column (4.6×250 mm), 1.0 mL/min, CH₃CN (10% to 70%, 30 min)], ¹H NMR: (300 MHz, D₂O) δ 7.45-6.98 (m, 7H), 4.32 (s, 2H), 3.92 (t, J=6.7 Hz, 2H), 3.87-3.81 (m, 2H), 3.82 (s, 3H), 2.65-2.60 (m, 1H), 2.48-2.43 (m, 1H), 1.90-1.83 (m, 2H), 1.54-1.45 (m, 1H), 1.21-1.03 (m, 3H), 0.95-0.91 (m, 1H), 0.68 (t, J=6.8 Hz, 3H), 0.66 (d, J=6.6 Hz, 3H); ³¹P NMR: (121 Hz, D₂O) δ 0.71, −3.91; MALDI-TOF MS, m/z 657.48 for [M−H]⁻ (calcd for C₂₈H₃₆ClN₂O₁₀P₂ 657.16).

23: HPLC, 26.84 min [Agilent Eclipse XOB-C18 column (4.6×250 mm), 1.0 mL/min, CH₃CN (10% to 70%, 30 min)], ¹H NMR: (300 MHz, D₂O) δ 7.47-7.06 (m, 7H), 4.34 (s, 2H), 3.94 (t, J=7.2 Hz, 2H), 3.84-3.82 (m, 2H), 2.67-2.60 (m, 1H), 2.51-2.44 (m, 1H), 1.87-1.78 (m, 2H), 1.54-1.48 (m, 1H), 1.19-1.06 (m, 3H), 0.95-0.91 (m, 1H), 0.71 (t, J=6.9 Hz, 3H), 0.67 (d, J=6.6 Hz, 3H); ³¹P NMR: (121 Hz, D₂O) δ 0.59, −3.84; MALDI-TOF MS, m/z 645.10 for [M−H]⁻ (calcd for C₂₇H₃₃ClFN₂O₉P₂ 645.08).

24: HPLC: 27.74 min [Agilent Eclipse XOB-C18 column (4.6×250 mm), 1.0 mL/min, CH₃CN (10% to 70%, 30 min)]; ¹H NMR: (300 MHz, D₂O) δ 7.40-7.11 (m, 7H), 3.90-3.82 (m, 2H), 3.72 (t, J=7.5 Hz, 2H), 3.18 (t, J=7.5 Hz, 2H), 2.98 (t, J=6.3 Hz, 2H), 2.56-2.49 (m, 1H), 2.39-2.31 (m, 1H), 1.87-1.81 (m, 2H), 1.48-1.44 (m, 1H), 1.18-1.01 (m, 3H), 0.94-0.89 (m, 1H), 0.75 (t, J=6.9 Hz, 3H), 0.66 (d, J=6.3 Hz, 3H); ³¹P NMR: (121 Hz, D₂O) δ 0.70, −3.91; MALDI-TOF MS, m/z 676.15 for [M−H]⁻ (calcd for C₂₈H₃₅Cl₂N₂O₉P₂ 676.13). Spectroscopic data for entries 25 and 26 of Table S1 were identical with those of entry 24.

### Determination of the Enantiomeric Excess of (S)—S18 and (R)-S19 by Analysis of Mosher's Esters.

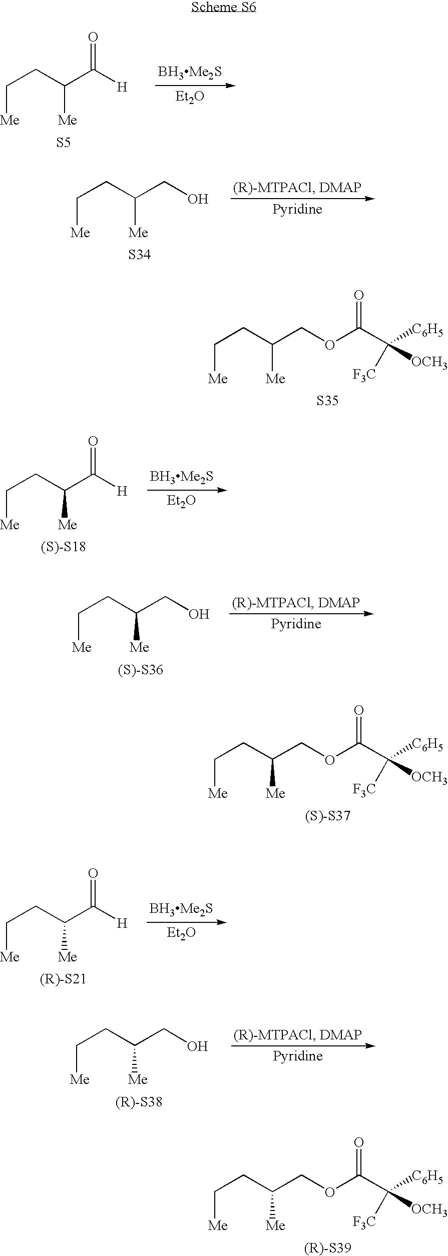

2-Methyl-1-pentanol (S34): To a cooled solution (0° C.) of the aldehyde S5 (0.34 g, 3.4 mmol) in Et₂O-pentane (3:2, 75 mL) under Ar was slowly added BH₃.Me₂S (1.27 g, 16.7 mmol, 5 equiv.) and the mixture stirred for 45 min. The mixture was quenched with aqueous 3 M HCl (20 mL) and stirred at room temperature for another 90 min. The aqueous phase was extracted with Et₂O (3×15 mL). The combined organic extracts were washed with aqueous Na₂SO₃ (30 mL), dried with magnesium sulfate and concentrated in vacuo. The residue was chromatographed on silica gel. Elution with pentane/diethyl ether (10:1, R_{f} 0.35) gave 0.28 g (82%) of S34 as a colorless oil. ¹H NMR: (300 MHz, CDCl₃) δ 3.54 (dd, J=10. 4, 6.2 Hz, 1H), 3.42 (dd, J=10.5, 6.4 Hz, 1H), 1.68-1.58 (m, 1H), 1.42-1.24 (m, 3H), 1.18-1.04 (m, 1H), 0.92-0.89 (m, 6H); ¹³C NMR: (75 MHz, CDCl₃) δ 68.63, 35.70, 35.61, 20.27, 16.74, 14.53; ESI-MS, m/z 103.1 for [M+H]⁺ (calcd for C₆H₁₅O, 103.1).

(S)-36: In the same manner as described above, (S)-S18 was converted into (S)-S36 (78%); [α]_{D}²⁰ −13.2° (c 2.00, MeOH), lit.⁶ [α]_{D}²⁰ −14.1°. NMR data were identical with those of S34.

(R)-38: In the same manner as described above, (R)-S21 was converted into (R) —S38 (72%); [α]_{D}²⁰ +12.3° (c 1.68, MeOH), lit.⁶ [α]_{D}¹⁸+14.1°. NMR data were identical with those of S34.

### Preparation of the Mosher Ester S35

To a DCM (2 mL) solution of 2-methyl-1-petanol (S34, 2.5 mg, 24.5 μmmol, 1.0 equiv.) were added 4-dimethylaminopyridine (DMAP, 0.6 mg, 5.2 μmol, 0.2 equiv.), pyridine (2.7 μL, 52.2 μmol, 2.0 equiv.) and (R)-(−)-α-Methoxy-α-trifluoromethylphenylacetyl chloride [(R)-MTPAC1, 5.7 μL, 33.9 μmmol, 1.3 equiv.] at room temperature, and stirring was continued for 1.5 h. After addition of N,N-dimethyl-1,3-propanediamine (3.6 μL, 52.2 μmol, 2.0 equiv.) and evaporation of solvent, the residue was passed through a disposable pipet packed with silica-gel (EA-Hexane, 1:30, R_{f}0.53) gave 4.2 mg (52%) of S35 as a colorless oil. Mixture of diastereomers; ¹H NMR: (300 MHz, CD₃OD) δ 7.52-7.49 (m, 4H), 7.44-7.41 (m, 6H), 4.28-4.06 (m, 4H), 3.47 (s, 6H), 1.87-1.80 (m, 2H), 1.38-1.22 (m, 6H), 1.17-1.11 (m, 2H), 0.93-0.85 (m, 12H); ¹³C NMR: (75 MHz, CD₃OD) δ 130.92, 129.60, 128.65, 72.25, 72.19, 36.61, 36.55, 33.60, 33.56, 21.01, 17.23, 17.20, 14.65; ESI-MS, m/z 319.2 for [M+H]⁺ (calcd for C₁₆H₂₂F₃O₃, 319.1).

(S)-S37: In the same manner as described above, (S)-S36 was converted into (S)-S37 (45%, de-95% by ¹H NMR); ¹H NMR: (300 MHz, CD₃OD) δ 7.52-7.47 (m, 2H), 7.46-7.41 (m, 3H), 4.27 (dd, J=10.8, 5.4 Hz, 1H), 4.12 (dd, J=10.5, 6.3 Hz, 1H), 3.48 (s, 3H), 1.87-1.80 (m, 1H), 1.39-1.25 (m, 3H), 1.16-1.10 (m, 1H), 0.92-0.90 (m, 6H); ¹³C NMR: (75 MHz, CD₃OD) δ 130.93, 129.61, 128.66, 72.26, 36.62, 33.57, 21.01, 17.18, 14.65; ESI-MS, m/z 319.2 for [M+H]⁺ (calcd for C₁₆H₂₂F₃O₃, 319.1).

(R)-S39: In the same manner as described above, (R)-S38 was converted into (R)-S39 (56%, de>95% by ¹H NMR); ¹H NMR: (300 MHz, CD₃OD) δ 7.53-7.46 (m, 2H), 7.44-7.39 (m, 3H), 4.20 (d, J=6.3 Hz, 1H), 4.17 (d, J=5.7 Hz, 1H), 3.46 (s, 3H), 1.86-1.80 (m, 1H), 1.37-1.22 (m, 3H), 1.16-1.09 (m, 1H), 0.91-0.89 (m, 6H); ¹³C NMR: (75 MHz, CD₃OD) δ 130.92, 129.60, 128.67, 72.19, 36.55, 33.60, 21.03, 17.24, 14.64; ESI-MS, m/z 319.2 for [M+H]⁺ (calcd for C₁₆H₂₂F₃O₃, 319.1).

Twenty-six different compounds were made and tested as inhibitors for Wip1 (Table 1) and the positions around the pyrrole ring were optimized for inhibition. In Table 1, the Wip1 inhibition constants (Ki) are shown for the compounds. For R1, the optimal group is a 2-chlorophenylphosphate and all compounds have this R1 group. Optimization then proceeded with R2. Several hydrophobic groups were examined, but alkyl chains with a branched methyl group were superior over straight chain alkyl groups. Ultimately, a 2-methylpentyl group was chosen as the ideal sidechain for this position. Optimization of the R3 group focused on finding the ideal distance between the phosphate group and the pyrrole core. As shown, this distance was clearly 3 methylene units. Next, for optimization at R4, it was determined that chloro-aromatic groups were ideal. Finally, each enantiomer of the 2-methylpentyl sidechain was prepared and the (S) enantiomer was clearly more active than the (R) enantiomer.

### Example 2

This example demonstrates the selectivity of the compounds in accordance with the invention.

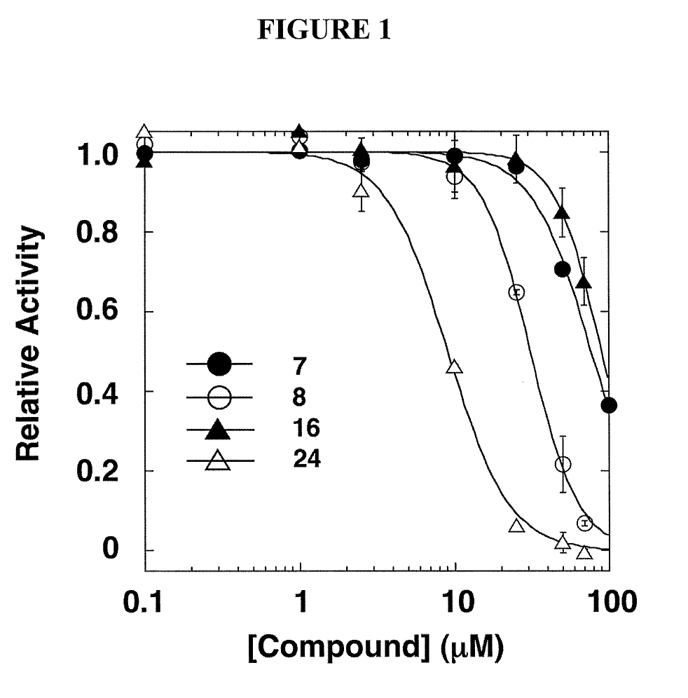

The selectivity of the compounds was determined. Certain compounds were tested to determine their selectivity in inhibiting Wip1, PP2Cα, and a K238D mutant of Wip1 according to the methods described above. As shown in Table 2, compounds 24, 25 and 26 were highly selective for Wip1, exhibiting no inhibition of the K238D mutant or PP2Cα. FIG. 1 also shows the relative activity of compounds 7, 8, 16, and 24 at various concentrations.

TABLE 2
Inhibition of PP2Cα and Wip1 Mutant
Phosphatase Activities by compounds

| | Kᵢ (μM) | | |
|---|---|---|---|
| | Wip1 WT | Wip1(K238D) | PP2Cα |
| Compound 24 | 5.7 ± 0.4 | NI | NI |
| Compound 26 (R) | 10 ± 1 | NI | NI |
| Compound 25 (S) | 4.7 ± 1 | NI | NI |

NT = no inhibition observed

The results show that the inventive compounds are effective and highly selective Wip1 inhibitors.

### Example 3

This example demonstrates the inhibitory effectiveness of compounds in accordance with the invention relative to Wip1.

The selectivity of three compounds in inhibiting Wip1 was tested on human breast cancer cell line MCF7 cells, the latter strongly expressing Wip1. These cells were treated with each compound, and cell lysates were collected after the indicated time point. 20 ug of total protein extracts were subjected to SDS-PAGE, and levels of phospho-p38 MAPK and p38 MAPK were examined by Western blot analysis using specific antibodies. Dimethyl sulphoxide (DMSO) and UV (25J/m2) treated cells were used as the negative and positive controls respectively. The results indicate that the diphosphate and monophosphate of compound 1A had relatively no effectiveness, while Compound 1A (prodrug) was effective.

### Example 4

This example illustrates a solution phase preparation of one exemplary Wip1 inhibitor prodrug (1A) contemplated by the present invention. This method of preparation permits the preparation of milligram quantities of the Wip1 inhibitor compounds.

### Synthesis of Wip1 Inhibitor Prodrug 1A

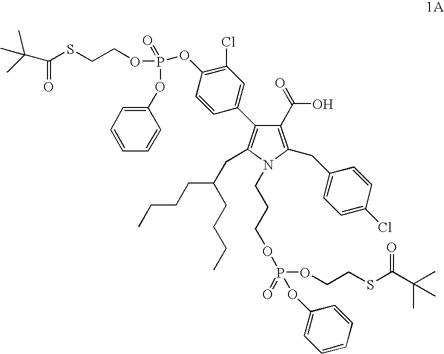

Preparation of β-ketoneamide 3 (Knoevenagel Reaction)

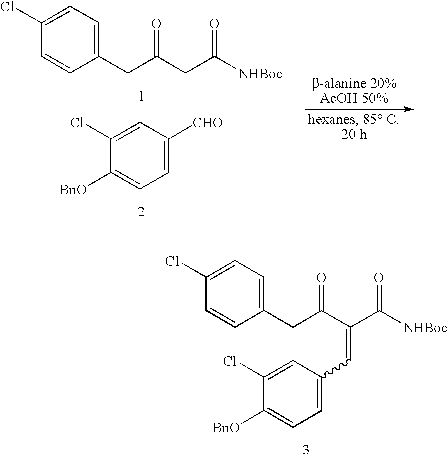

To a suspension of β-ketoneamide 1 (490 mg, 1.57 mmol), benzaldehyde 2 (352 mg, 1.43 mmol), β-alanine (25 mg, 0.29 mmol) in hexanes (15 mL), glacial acetic acid was added (43 mg, 0.041 mL, 0.72 mmol). The resulting suspension was heated to reflux with removal of water for 20 hours (Dean-Stark apparatus). If necessary, additional hexanes and glacial acetic acid may be added. The reaction mixture was cooled to room temperature (rt). Saturated aq. NaHCO₃ solution was added to the reaction mixture and extracted with ethyl acetate (2×10 ml). The extract was dried over Na₂SO₄. Evaporation of the solvents and purification of the residue over a silica gel column using hexane/ethyl acetate (5:2) as eluent provided 3 as light yellow solid (685 mg, 81%). ¹H NMR (300 MHz, CDCl₃): δ 7.03-7.77 (m, 8H), 5.36 (s, 2H), 4.18 (s, 2H), 1.50 (s, 9H). ¹³C NMR (75 MHz, CDCl₃): δ 194.68, 156.00, 150.43, 135.76, 132.89, 132.33, 131.80, 129.76, 129.25, 128.66, 128.59, 128.54, 128.20, 127.68, 127.02, 126.96, 126.52, 126.28, 123.67, 113.77, 83.00, 70.79, 27.74.

### Preparation of β-dibutylpropanal 4

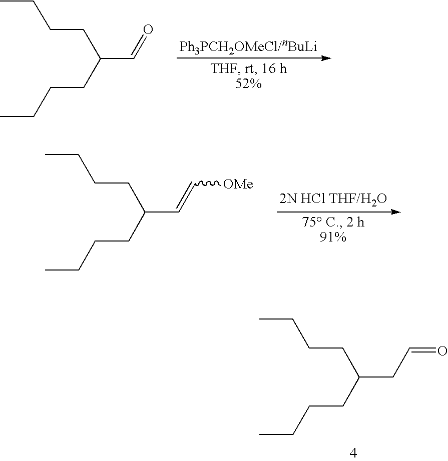

To the stirred suspension of methoxymethyltripgenylphosphonium chloride (2.62 g, 7.68 mmol) in dry THF (10 mL) at 0° C. was added a solution of nBuLi (1.6 M in hexanes, 5.28 mL, 8.44 mmol) dropwise and the resulting dark red solution was stirred for 1 h at rt. The solution was cooled to 0° C., and a solution of dibutylacetyaldehyde (400 mg, 2.56 mmol) in THF (1 mL) was added. The mixture was slowly warmed to rt and stirred overnight (16h). Saturated aq. NH₄Cl solution was added to the reaction mixture and extracted with diethyl ether (2×10 ml). The extract was dried over Na₂SO₄. Evaporation of the solvents and purification of the residue over silica gel column using hexane as eluent finished the enol ether as colorless liquid (245 mg, 52%). 1:1 E/Z isomers. ¹H NMR (300 MHz, CDCl₃): δ 6.34 (d, 1H, J=12.5 Hz E-isomer), 6.02 (d, 1H, J=6.0 Hz, Z-isomer), 4.58 (dd, 1H), 4.19 (dd, 1H), 3.62 (s, 3H), 3.61 (s, 3H), 2.60 (m, 1H), 1.84 (m, 1H), 1.56-1.03 (m, 18H).

A stirring solution of the enol ether (245 mg, 1.33 mmol) in THF (10 mL) and 2N of HCl (1.2 mL) was heated at 75° C. for 2 h. The reaction solution was cooled to rt and was diluted with water (5 mL) and extracted with diethyl ether (2×10 ml). The combined organic extract was washed with aq. NaHCO3 and brine, and dried over Na₂SO₄, evaporation of the solvent gave β-dibutylpropanal 4 (205 mg, 91%). ¹H NMR (300 MHz, CDCl₃): δ 9.87 (t, 1H), 2.44 (dd, 2H), 2.03 (m, 1H), 1.39 (m, 12H), 1.01 (m, 6H).

### Preparation of 1,4-diketone isomers 5 (Stetter Reaction)

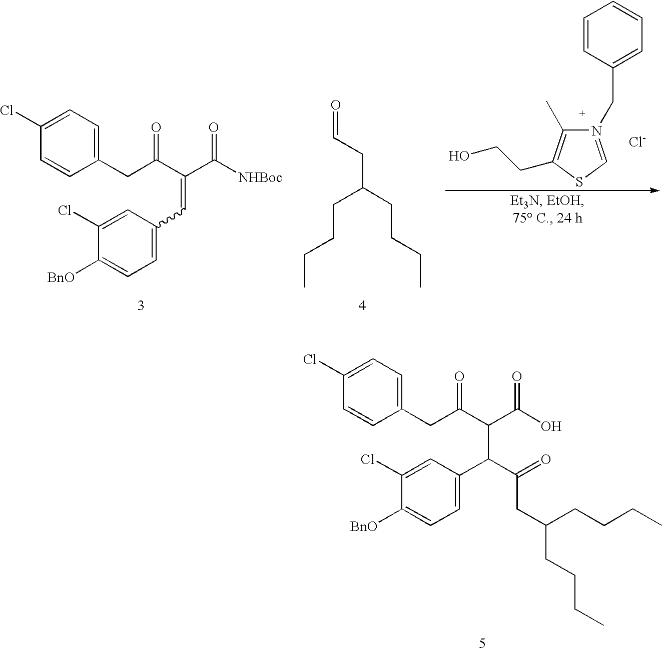

To a solution of β-ketoneamide 3 (500 mg, 0.93 mmol), 3-benzyl-5-(2-hydroxyethyl)-4-methylthiazolium chloride (126 mg, 0.47 mmol) in anhydrous ethanol (4 mL) was add a solution of β-dibutylpropanal 4 (174 mg, 1.02 mmol) in ethanol (1 mL), followed by the addition of triethylamine (94 mg, 0.93 mmol). The resulting solution was stirred and heated at 80° C. for 24 hours. Saturated aq. NH₄Cl solution was added to the reaction mixture and extracted with ethyl acetate (2×10 ml). The extract was dried over Na₂SO₄. Evaporation of the solvents and purification of the residue over silica gel column using hexane/ethyl acetate (5:1) as eluent finished 5 as colorless oil (220 mg, 39%). ¹H NMR (300 MHz, CDCl₃): δ 7.59-7.31 (m, 10H), 7.12-7.05 (m, 2H), 5.26 (s, 2H), 4.48 (d, 1H, J=2.7 Hz), 4.40-4.32 (q, 2H), 3.55 (d, 1H, J=2.7 Hz), 2.60-2.52 (dd, 1H, J=14.1, 10.5 Hz), 2.22-2.16 (dd, 1H, J=14.1, 4.5 Hz), 1.75 (m, 1H), 1.42-0.97 (m, 12H), 0.93-0.85 (m, 6H).

### Preparation of pyrrole 6 (Paal-Knorr Reaction)

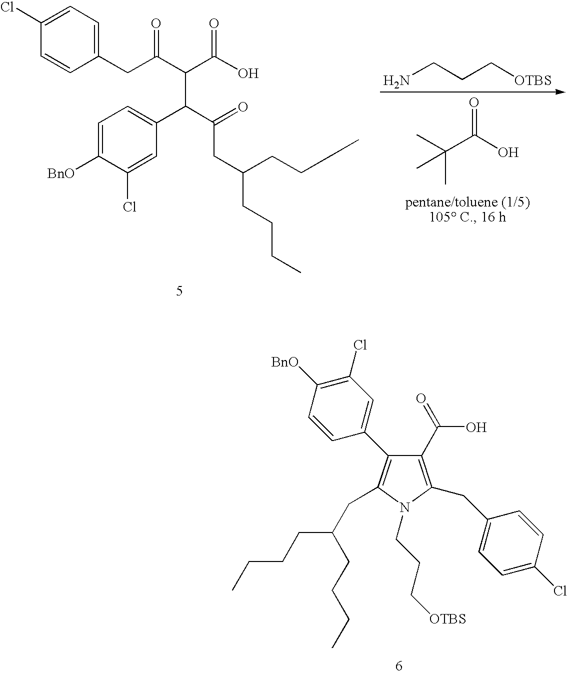

TBS protected 3-aminopropanol (60 mg, 0.32 mmol) in toluene (2 mL) was added to the solution of carboxylic acid 5 (130 mg, 0.21 mmol) and trimethylacetic acid (15 mg, 0.15 mmol) in the mixed solvents of heptane (5 mL) and toluene (23 mL). Anhydrous Na₂SO₄ (5 g) was added. The mixture was reflux at 105° C. for 16 h. The reaction solution was cooled to room temperature, Na₂SO₄ was filtered off Evaporation of the solvents and purification of the residue over silica gel column using hexane/ethyl acetate (5:1) as eluent afforded 6 as colorless oil (150 mg, 93%). ¹H NMR (300 MHz, CDCl₃): δ 7.59-7.04 (m, 12H), 5.27 (s, 2H), 4.78 (d, 1H, J=2.7 Hz), 3.81 (t, 2H), 3.55-3.47 (m, 2H), 3.30 (d, 1H, J=2.7 Hz), 2.60-2.52 (dd, 1H, J=14.1, 10.5 Hz), 2.25-2.19 (dd, 1H, J=14.1, 4.5 Hz), 1.87-1.81 (m, 2H), 1.74 (m, 1H), 1.34-0.94 (m, 12H), 0.95 (s, 9H), 0.94-0.85 (m, 6H), 0.16 (d, 6H). ¹³C NMR (75 MHz, CDCl₃): δ 202.73, 179.06, 165.79, 153.74, 139.78, 136.59, 134.50, 134.37, 130.89, 129.87, 129.72, 128.93, 128.86, 128.29, 127.67, 127.26, 124.16, 114.73, 71.18, 61.50, 61.26, 48.50, 37.62, 36.04, 34.31, 33.69, 33.26, 32.24, 28.98, 28.13, 26.15, 23.13, 22.75, 14.27, 14.23, −5.17, −5.20.

### Preparation of Pyrrole 7

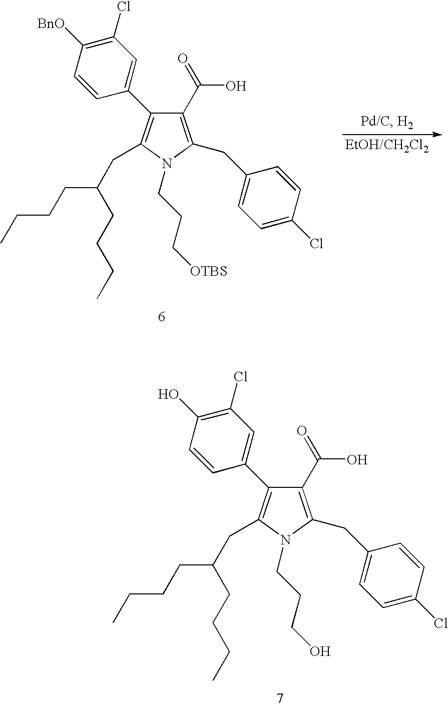

Pyrrole 6 (180 mg) was dissolved in methylene chloride (2 mL) and was added to the Parr flask containing 10% Pd/C (50 mg) and mixed solvents of 95% ethanol (3 mL) and methylene chloride (3 mL). The reaction was performed under hydrogen (30 psi) on Parr apparatus for 24 h. Pd/C was filtered off. Evaporation of the solvents and purification of the residue over silica gel column using methylene chloride/methanol (10:1) as eluent afforded 7 as colorless oil (120 mg, 89%), which solidifies upon standing in freezer. ¹H NMR (300 MHz, CDCl₃): δ 7.54-7.10 (m, 7H), 5.92 (s, br, 1H), 4.78 (d, 1H, J=2.7 Hz), 3.73 (t, 2H), 3.60-3.54 (m, 2H), 3.35 (d, 1H, J=2.7 Hz), 2.97 (m, 1H), 2.60-2.53 (dd, 1H, J=14.1, 10.5 Hz), 2.27-2.21 (dd, 1H, J=14.1, 4.5 Hz), 1.85-1.73 (m, 3H), 1.35-1.01 (m, 12H), 0.92-0.86 (m, 6H), 0.16 (d, 6H). ¹³C NMR (75 MHz, CDCl₃): δ 202.92, 179.88, 167.38, 151.62, 139.74, 134.48, 133.40, 130.86, 129.69, 129.46, 128.98, 128.89, 128.69, 127.94, 121.01, 117.36, 61.35, 59.68, 48.64, 36.92, 36.10, 34.38, 33.74, 33.28, 32.23, 28.98, 28.16, 23.10, 22.75, 14.24, 14.20.

### Preparation of Phosphate Prodrug 1a

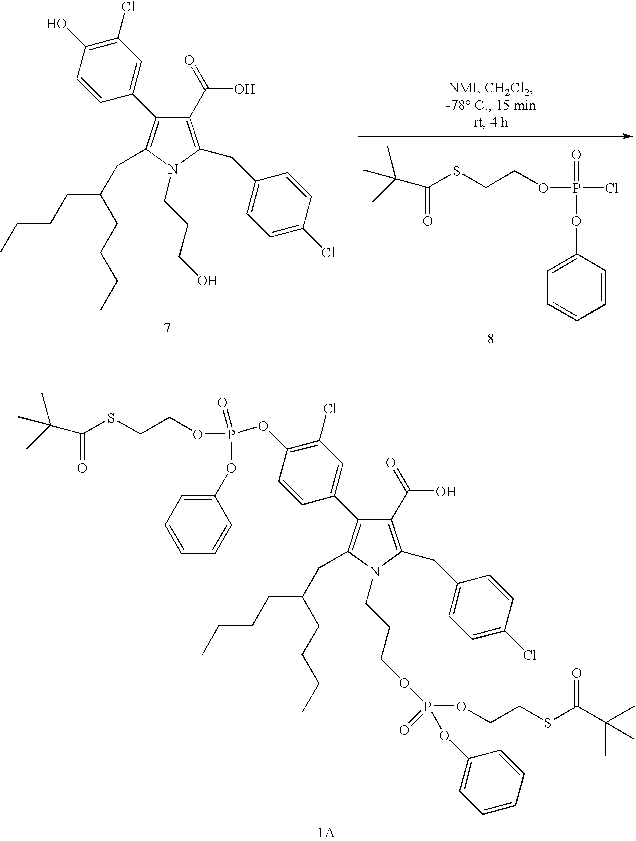

Chlorophosphate 8 (102 mg, 0.30 mmol) in methylene chloride (0.5 mL) was added to the solution of pyrrole 7 (62 mg, 0.11 mmol) in methylene chloride (2 mL). The mixture was cooled to −78° C. and N-methylimidazole (54 mg, 0.052 mL, 0.66 mmol) was added. The reaction was kept at −78° C. for 15 min and then room temperature for 4 h. The reaction solution was diluted with methylene chloride and washed with HCl (0.1M, 10 mLx 3). The organic layer was dried over Na₂SO₄. Evaporation of the solvents and purification of the residue over silica gel column using methylene chloride/methanol (30:1) as eluent afforded 1A as colorless oil (89 mg, 70%). ¹H NMR (300 MHz, CDCl₃): δ 7.53-7.20 (m, 17H), 4.81 (d, 1H, J=2.7 Hz), 4.51-4.44 (q, 2H), 4.24-4.00 (m, 4H), 3.53-3.47 (m, 2H), 3.31-3.21 (m, 5H), 2.63-2.55 (dd, 1H, J=14.1, 10.5 Hz), 2.21-2.15 (dd, 1H, J=14.1, 4.5 Hz), 1.73 (m, 1H), 1.33 (d, 18H), 1.39-1.00 (m, 12H), 0.90-0.85 (m, 6H). HRMS (ES+) calcd for C₅₇H₇₄NCl₂O₁₂P₂S₂ (M+1) 1160.3505, found 1160.3478.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound comprising a ring structure and at least five functional groups bonded thereto, wherein each functional group is bonded to a different ring atom, and wherein the at least five functional groups comprise: (a) first (R₁) and second (R₃) moieties each comprising a phosphate group wherein these first and second moieties are separated by at least one ring atom; (b) first (R₂) and second (R₄) hydrophobic groups, wherein the first and second hydrophobic groups are separated by at least one ring atom, and wherein the first hydrophobic group is bonded to a ring atom located between the ring atoms to which the first (R₁) and second (R₂) moieties are bonded; and an amide or carboxylic acid (R₅).

2. The compound according to claim 1, wherein the ring is heterocyclic.

3. The compound according to claim 2, wherein the ring comprises at least one nitrogen atom or sulfur atom and the remaining ring atoms are carbon.

4. The compound according to claim 3, wherein one of R₁, R₂, R₃ or R₄ is bonded to the nitrogen or sulfur atom.

5. The compound according to claim 4, wherein the first and second hydrophobic groups, R₂ and R₄, respectively, may be the same or different, and desirably comprise alkyls, alkenyls, alkynyls, heteroalkyls, cycloalkyls, heterocycloalkyls, acyls, aryls, heteroaryls, amino acids, or peptides comprising between 2 and 5 amino acids.

6. The compound according to claim 4, wherein the first and second moieties which each comprise a phosphate group, R₁ and R₃, respectively, may be the same or different, and desirably comprise alkyls, alkenyls, alkynyls, heteroalkyls, cycloalkyls, heterocycloalkyls, acyls, aryls or heteroaryls.

7. The compound according to claim 5, wherein R₂ is non-cyclic and R₄ comprises a cyclic structure.

8. The compound according to claim 6, wherein R₁ comprises an aryl and R₃ comprises an alkyl, alkenyl or alkynyl.

9. The compound according to claim 7, wherein R₂ is a C₁-C₁₂ alkyl, alkenyl or alkynyl and R₄ comprises an aryl.

10. The compound according to claim 8, wherein R₁ comprises a 5- or 6-membered aryl and R₃ comprises a C₁₋₆ alkyl, alkenyl or alkynyl.

11. The compound according to claim 9, wherein R₂ is a branched C₁-C₈ alkyl, alkenyl or alkynyl.

12. The compound according to claim 11, wherein R₂ comprises a branched C₄-C₆ alkyl, alkenyl or alkynyl, R₄ comprises an aryl which is linked to the ring by a C₁₋₄ alkyl, alkenyl or alkynyl, and the ring comprises one nitrogen atom and the remaining ring atoms are carbon.

13. The compound according to claim 10, wherein R₁ comprises phenyl and R₃ comprises a C₁₋₃ alkyl, alkenyl or alkynyl.

14. The compound according to claim 12, wherein R₂ comprises methylpropyl or methylpentyl and R₄ comprises phenyl linked to the ring via an ethyl group.

15. The compound according to claim 13, wherein R₁ comprises a halogen-substituted phenyl and R₃ comprises propyl, propenyl or propynyl.

16. The compound according to claim 1, wherein the compound comprises a 5-membered ring structure.

17. The compound according to claim 16, wherein the compound has the structure (I): 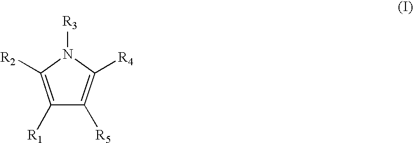
wherein the first and second hydrophobic groups, R₂ and R₄, respectively, may be the same or different, and desirably comprise alkyls, alkenyls, alkynyls, heteroalkyls, cycloalkyls, heterocycloalkyls, acyls, aryls, heteroaryls, amino acids, or peptides comprising between 2 and 5 amino acids, and wherein the first and second moieties which each comprise a phosphate group, R₁ and R₃, respectively, may be the same or different, comprise alkyls, alkenyls, alkynyls, heteroalkyls, cycloalkyls, heterocycloalkyls, acyls, aryls or heteroaryls.

18. The compound according to claim 17, wherein R₂ is non-cyclic, R₄ comprises a cyclic structure, R₁ comprises an aryl, and R₃ comprises an alkyl, alkenyl or alkynyl.

19. The compound according to claim 18, wherein R₂ is a C₁-C₁₂ alkyl, alkenyl or alkynyl, R₄ comprises an aryl, R₁ comprises a 5- or 6-membered aryl, and R₃ comprises a C₁₋₆ alkyl, alkenyl or alkynyl.

20. The compound according to claim 19, wherein R₂ comprises a branched C₄-C₆ alkyl, alkenyl or alkynyl, R₄ comprises an aryl which is linked to the ring by a C₁₋₄ alkyl, alkenyl or alkynyl, R₁ comprises phenyl, R₃ comprises a C₁₋₃ alkyl, alkenyl or alkynyl, and the ring comprises one nitrogen atom and the remaining ring atoms are carbon.

21. The compound according to claim 20, wherein R₂ comprises methylpropyl or methylpentyl, R₄ comprises phenyl linked to the ring via an ethyl group, R₁ comprises a halogen-substituted phenyl, and R₃ comprises propyl, propenyl or propynyl.

22. The compound according to claim 1, wherein the compound comprises a 6-membered ring structure.

23. The compound according to claim 22, wherein the 6-membered ring structure is aromatic.

24. The compound according to claim 23, wherein the compound has the structure (II): 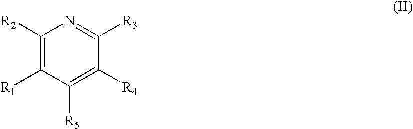
wherein the first and second hydrophobic groups, R₂ and R₄, respectively, may be the same or different, and desirably comprise alkyls, alkenyls, alkynyls, heteroalkyls, cycloalkyls, heterocycloalkyls, acyls, aryls, heteroaryls, amino acids, or peptides comprising between 2 and 5 amino acids, wherein the first and second hydrophobic groups, R₂ and R₄, respectively, may be the same or different, and desirably comprise alkyls, alkenyls, alkynyls, heteroalkyls, cycloalkyls, heterocycloalkyls, acyls, aryls, heteroaryls, amino acids, or peptides comprising between 2 and 5 amino acids, and wherein the first and second moieties which each comprise a phosphate group, R₁ and R₃, respectively, may be the same or different, comprise alkyls, alkenyls, alkynyls, heteroalkyls, cycloalkyls, heterocycloalkyls, acyls, aryls or heteroaryls.

25. The compound according to claim 24, wherein R₂ is non-cyclic, R₄ comprises a cyclic structure, R₁ comprises an aryl, and R₃ comprises an alkyl, alkenyl or alkynyl.

26. The compound according to claim 25, wherein R₂ is a C₁-C₁₂ alkyl, alkenyl or alkynyl, R₄ comprises an aryl, R₁ comprises a 5- or 6-membered aryl, and R₃ comprises a C₁₋₆ alkyl, alkenyl or alkynyl

27. The compound according to claim 26, wherein R₂ comprises a branched C₄-C₆ alkyl, alkenyl or alkynyl, R₄ comprises an aryl which is linked to the ring by a C₁₋₄ alkyl, alkenyl or alkynyl, R₁ comprises phenyl, R₃ comprises a C₁₋₃ alkyl, alkenyl or alkynyl, and the ring comprises one nitrogen atom and the remaining ring atoms are carbon.

28. The compound according to claim 27, wherein R₂ comprises methylpropyl or methylpentyl, R₄ comprises phenyl linked to the ring via an ethyl group, R₁ comprises a halogen-substituted phenyl, and R₃ comprises propyl, propenyl or propynyl.

29. A prodrug of a compound according to claim 1.

30. A method of inhibiting the activity of a Wip1 protein in a cell comprising providing a cell comprising a Wip1 protein, and contacting the cell with a compound according to claim 1, wherein the activity of the Wip1 protein in the cell is inhibited.

31. The method of claim 30, wherein the cell is a mammalian cell.

32. The method of claim 31, wherein the cell is a human cell.

33. The method of claim 32, wherein the cell is a cancer cell.

34. The method of claim 33, wherein the cancer is selected from the group consisting of breast cancer, neuroblastoma, ovarian cancer, and colon cancer.

35. The method of claim 30, wherein the phosphatase activity of a Wip1 protein is inhibited.

36. A pharmaceutical composition comprising a carrier and a compound according to claim 1.

37. The composition according to claim 36, wherein the carrier is a pharmaceutically-acceptable carrier.

38. A method for treating cancer comprising administering to a mammal in need of cancer therapy a prodrug of a compound according to claim 1.

39. The method according to claim 38, wherein the cancer is selected from the group consisting of breast cancer, neuroblastoma, ovarian cancer, and colon cancer.

40. The compound according to claim 1, wherein the compound is selected from the group consisting of: 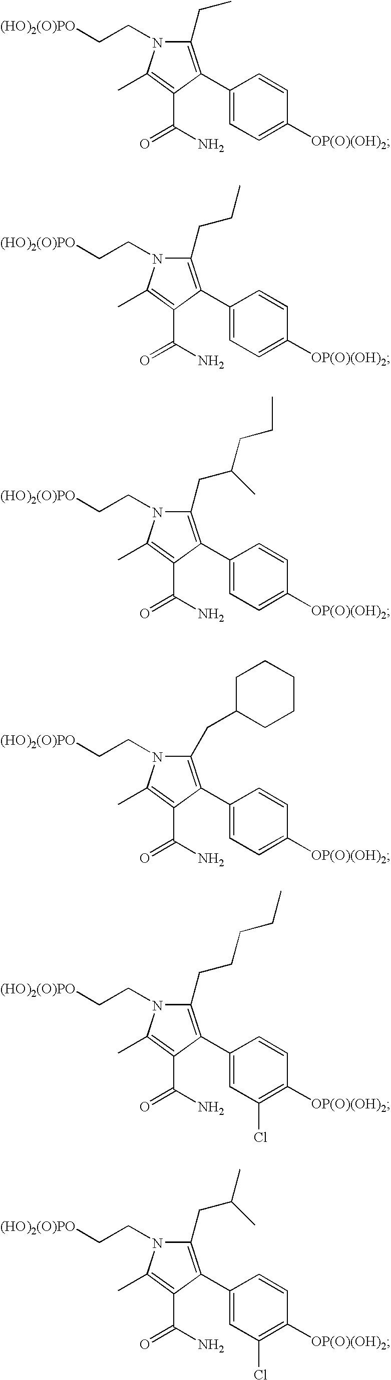 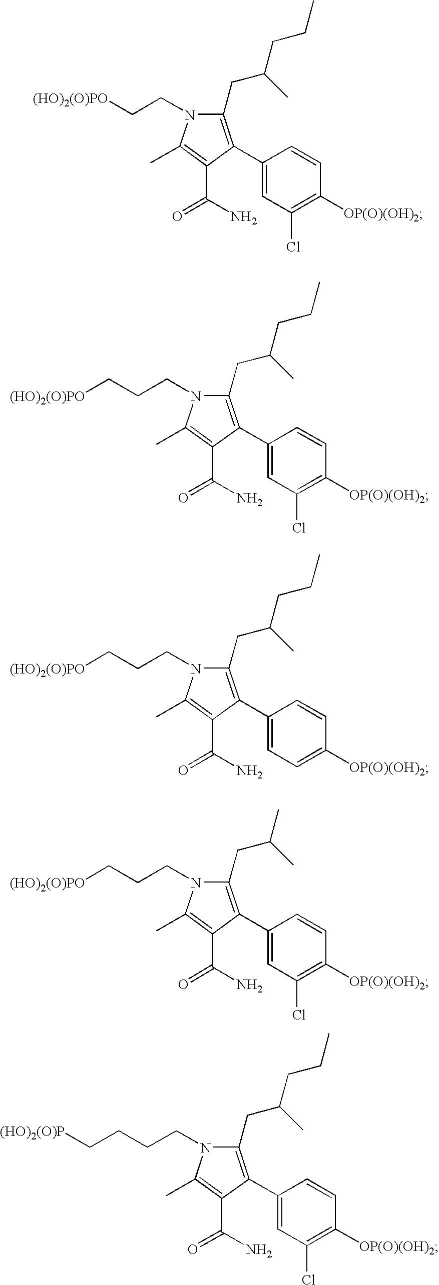 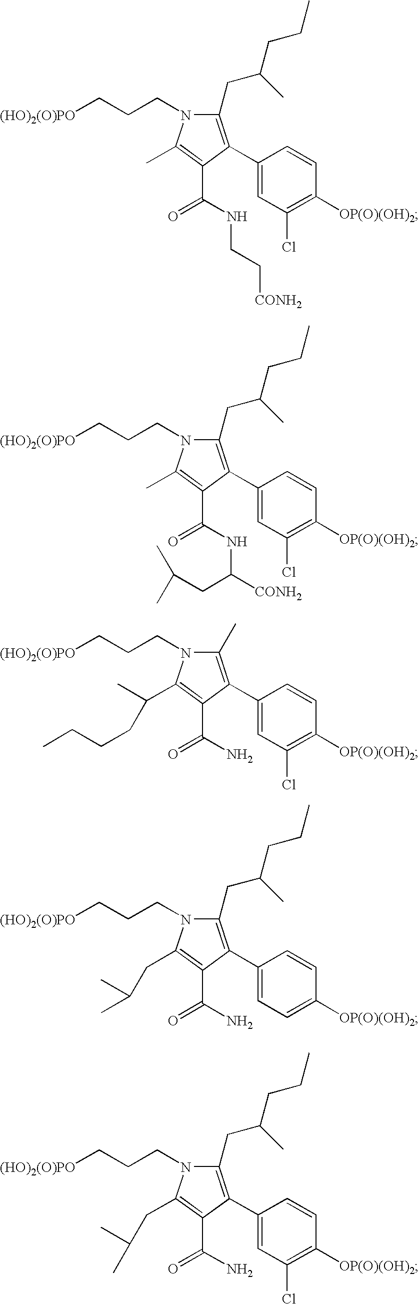 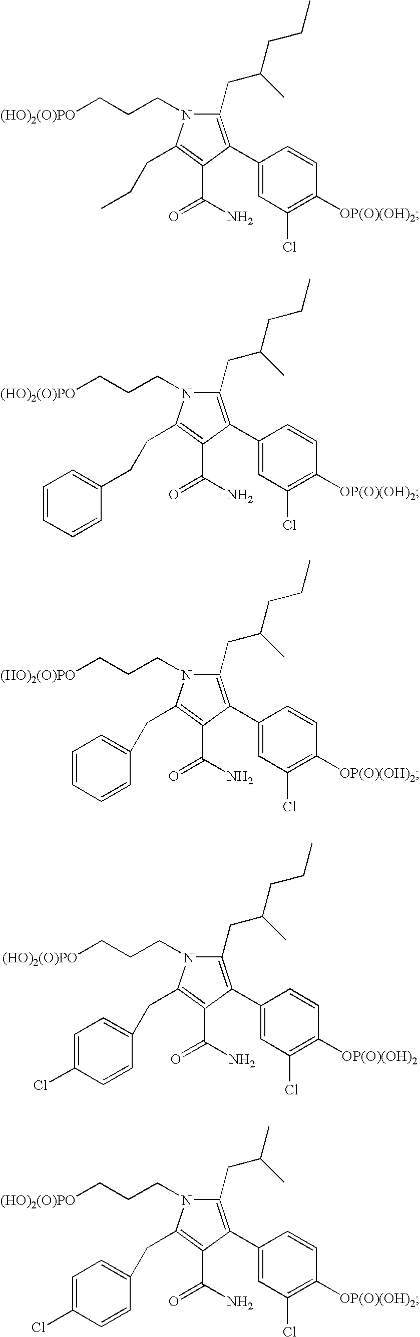 [chemical structure]
